SEQUENCE LISTING (14 sequences)

SEQ ID NO: 1 - DNA, 408 bp, Homo sapiens
tctccgcctccttcttcatcatggtcatgatccccggggtgggctacagccttcaggcccaccccagttccctccctaccgcggaatgatgccgcctttcatgtatcccccatatctcccgttccctccgccctatggaccccaggggccttaccgataccccactcctgatgggcccagccgttttccccgtgtggcgggcccccgaggctcagggccaccaatgcgcttagtagagcctgtgggtcatccctctattctcaaagaggataatctcaaagagtttgatcagttggatcaggagaatgatgatggttgggcaggggcccatgaagaggttgactacactgaaaagctcaagttcagcgatgaagaaggaggcggagagggagatgtattgggagaagg

SEQ ID NO: 2 - protein, 9 aa, Homo sapiens
SPPPSSSWS

SEQ ID NO: 3 - DNA, 19 bp, Homo sapiens
tgcgcttagtagagcctgt

SEQ ID NO: 4 - protein, 46 aa, Homo sapiens
SPGWATAFRPTPVPSLPRNDAAFHVSPISPVPSALWTPGALPIPHS

SEQ ID NO: 5 - protein, 33 aa, Homo sapiens
WAQPFSPCGGPPRLRATNALSRACGSSLYSQRG

SEQ ID NO: 6 - protein, 4 aa, Homo sapiens
SQRV

SEQ ID NO: 7 - protein, 6 aa, Homo sapiens
SVGSGE

SEQ ID NO: 8 - protein, 6 aa, Homo sapiens
WLGRGP

SEQ ID NO: 9 - protein, 2 aa, Homo sapiens
RG

SEQ ID NO: 10 - protein, 2 aa, Homo sapiens
LH

SEQ ID NO: 11 - protein, 6 aa, Homo sapiens
KAQVQR

SEQ ID NO: 12 - protein, 12 aa, Homo sapiens
RRRRRGRCIGRR

SEQ ID NO: 13 - DNA, 22 bp, Artificial Sequence
  Description of Artificial Sequence Primer
ccttcctcccaatacatctccc

SEQ ID NO: 14 - DNA, 20 bp, Artificial Sequence
  Description of Artificial Sequence Primer
tctccgcctccttcttcatc

(19) United States
(12) Patent Application Publication
(10) Pub. No.: US 2005/0136434 A1
(43) Pub. Date: Jun. 23, 2005

(54) Isolated heat-inducible cell surface protein and hyperthermia-based tumor immunotargeting therapy

(76) Inventor: Mai Xu, St. Louis, MO (US)

Correspondence Address: Patrick W. Rasche;Armstrong Teasdale LLP, Suite 2600, One Metropolitan Square, St. Louis, MO, 63102, US

(21) Appl. No.: 10/916,923

(22) Filed: Aug. 12, 2004

Related U.S. Application Data
(60) Provisional application No. 60/494,398, filed on Aug. 12, 2003.

(51) Int. Cl. C12Q 1/68; G01N 33/574; C07H 21/04; C07K 16/30; C07K 14/705

(52) U.S. Cl. 435/6; 435/7.23; 435/69.1; 435/320.1; 435/325; 530/350; 530/388.8; 536/23.5

(57) ABSTRACT

An isolated, purified and characterized gene comprising a polynucleotide having a sequence shown in Seq. Id. No. 1. An isolated, purified and characterized protein comprising a polypeptide having a sequence shown in Seq. Id. Nos. 2 & 4-12, respectively. In an aspect, a method of activating a gene or inducing the gene encoded protein comprises intentionally applying finite heat for a time and in an amount effective to living cells, tissues, organs or a whole body to cause and thereby causing activation of a gene comprising a polynucleotide having a sequence shown in Seq. Id. No. 1 and production of an encoded protein comprising a polypeptide having a sequence shown in Seq. Id. Nos. 2 & 4-12, respectively. In an aspect, a method of stress including hyperthermia-based tumor immunotargeting therapy for the treatment of neoplasm and pre-cancerous lesions.

This application claims the benefit of U.S. Provisional Patent Application Ser. No. 60/494,398 filed Aug. 12, 2003 which is incorporated herein in its entirety by reference.

### FIELD OF THE INVENTION

This invention relates to cancer therapy. More specifically the invention relates to diagnosis and treatment of benign and malignant neoplasm as well as pre-cancerous lesions in a living mammal.

### BACKGROUND OF THE INVENTION

Cancer (malignant neoplasm) is a leading killer disease of mankind and the number two killer of people in the U.S. Each year in the U.S. more than a million people are diagnosed with cancer and half of those will ultimately die from cancer. Cancer generally manifests itself as clinically apparent tumors in tissue which are generally detectable by one or more of PET scans, SPECT scans, ultrasound, magnetic resonance imaging (MRI), CT scans, X-ray imaging and digital mammography. After a clinical diagnosis of cancer is made, a first line treatment is begun.

First line treatments include the conventional modalities of surgery, radiotherapy, and chemotherapy. These treatments can be used individually or in combination with one another. Despite some increasing success, these conventional modalities are not always effective to the degree desired, and intensive research has continued in an attempt to identify more efficacious therapies. Most forms of cancer still remain refractory to these conventional treatment modalities. The major limitation of these present modalities is the unfortunate narrow therapeutic index between normal and malignant neoplastic cells. Therefore it is highly desired to increase the killing of cancer cells and selectively preserve the life of normal cells.

Targeting therapies are specifically directed or targeted to cancer cells and are less likely to affect normal cells or normal tissues than do conventional therapies do such as chemotherapy and radiotherapy.

Antibody-guided immunotargeting therapy is one type of targeting therapies. Key elements of antibody-guided immunotargeting therapy include 1) antigen specificity to tumor or target cells, 2) antibodies specificity to the antigen expressed on tumor or target cells, and 3) efficiency of delivering conjugated complex of antibodies and anti-cancer agents to the cells in a hypoxic area of solid tumor mass.

However tumor specificity of antigen or molecules is targeted, heterogeneity of antigen expression, and efficiency of delivering conjugated complex of antibodies and anti-cancer agents to the cells in a hypoxic area of solid tumor mass remain major obstacles for the use of antibody-guided tumor immunotargeting therapy in clinical practice.

To improve tumor specificity of targeted antigens, a tremendous effort has been made and continues to be made to identify tumor specific antigens and develop smarter “magic bullets”, i.e., conjugated complex of antibody against tumor specific or associated antigens and cytotoxic or anti-cancer agents. Presently, most antigens identified are tumor-associated antigens, which are expressed in both tumor and normal cells. For example TAG72 antigen was reportedly expressed more prominent in the mucosa adjacent to the tumor than in the tumor tissue and expressed heterogeneously on tumor cells in colon cancer. The antibody CC49 against TAG72 antigen has been used in clinics for cancer treatment. Mixed therapeutic results from CC49 immunotargeting therapy for different kind of cancer has been reported.

Recently Herceptin (generic name, Trastuzumab), Rituxan and Compath have shown promise in clinical trials for the treatment of breast cancer, malignant lymphoma and leukemia respectively. However, human epithelial growth factor receptor 2 (HER2), target molecule of Herceptin (antibody), was expressed only in one third of patients with breast cancer. Rituxan and Compath are antibodies that bind to CD20 and CD52 antigen on the surface of lymphoma and leukemia cells respectively as well as on normal lymphocyte. Those drugs, Rituxan and Compath, kill lymphoma and leukemia cells but also deplete normal lymphocyte as well.

The weaknesses of current tumor immunotargeting therapy that need to be improved include tumor specificity, homogeneity (antigen expression heterogeneously on targeted cells) and efficiency of delivering “magic bullet” to cells in a hypoxic area of solid tumor masses. Smarter “magic bullets” and an enhanced delivery system remain urgently needed.

### BRIEF DESCRIPTION OF THE INVENTION

In one aspect, this discovery provides an isolated and characterized functional gene comprising a polynucleotide having a sequence shown in Seq. Id. No. 1.

In an aspect, an isolated and characterized protein comprises a polypeptide having a sequence such as a (HICSP) shown in Seq. Id. No. 2. In an aspect, this protein is a target protein of an antibody of this discovery (HICSP—heat induced cell surface protein.

In an aspect, a method of activating a gene or inducing production and/or translocation of a gene encoding a protein comprises applying finite heat to living cells, tissues, organs or a whole body for a time and in an amount effective manner to cause activation of a gene comprising a polynucleotide having a sequence shown in Seq. Id. No. 1. In an aspect production ensues of an encoded protein comprising a polypeptide having a sequence shown in Seq. Id. No. 2.

In an aspect, a method of activating a gene or inducing production and/or translocation of the gene encoded protein comprises effectively applying an effective stress-inducing amount of an agent selected from the group consisting of mammalian stress-causing chemicals and biological substances to living cells, tissues, organs or a whole living mammalian body to cause activation of a gene comprising a polynucleotide having a sequence shown in Seq. Id. No. 1. In an aspect production ensues of an encoded protein comprising a polypeptide having a sequence shown in Seq. Id. No. 2.

In an aspect, isolated, characterized and purified functional recombinant or transfected cells having stability and competent integrated in its genome a gene comprise a polynucleotide having a sequence shown in Seq. Id. No. 1 useful as a cell model for the study of a gene function. In an aspect, the gene having Seq. Id. No. 1 encodes a protein comprising a polypeptide having a sequence shown in Seq. Id. No. 2.

In an aspect, E. Coli XL 10-gold Ultra-competent cells (QiaGen) have been transfected with the cloned vector contain a polynucleotide having a sequence shown in Seq. Id No. 1.

In another aspect, an isolated, purified and characterized biological marker useful for identifying the presence of and specifying the location of a tumor locus in tissue comprises a gene comprising a polynucleotide having a sequence shown in Seq. Id. No. 1. In another aspect, a gene comprising a polynucleotide having a sequence shown in a sequence shown in Seq. Id. No. 1 is used as a tumor locating agent in a mammal.

In an aspect, an isolated, purified and characterized biological marker useful for specifying the location of a tumor locus in tissue comprises a protein comprising a polypeptide having a sequence shown in Seq. Id. No. 2. In another aspect, antibodies are provided against a protein comprising polypeptide having a sequence shown in Seq. Id. No. 2 as a tumor locating agent in a living mammal.

In an aspect, an antibody selected from one of monoclonal and polyclonal antibodies recognizing a protein comprising a polypeptide having a sequence shown in Seq. Id. No. 2.

In an aspect, an isolated and characterized conjugated antibody recognizing a protein comprising a polypeptide having a sequence shown in Seq. Id. No. 2 and further optimally comprising an anti-cancer agent. In an aspect, the antibody binds to the protein.

In an aspect, a pharmaceutical composition comprising a vaccine containing a protein comprising a polypeptide having a sequence shown in Seq. Id. No. 2 and an optimally suitable pharmaceutically acceptable carrier.

In an aspect, a pharmaceutical kit comprising a container housing an antibody recognizing a protein comprising a polypeptide having sequence shown in Seq. Id. No. 2 and optionally a suitable pharmaceutical carrier therewith.

A method of therapeutically treating a living mammal which comprises administering an anti-tumor agent or a functional derivative thereof, alone or in combination with a tumor-specific antibody or tumor-directing agent to the living mammal based upon determining the presence of a protein in the living animal, the protein comprising a polypeptide having sequence shown in Seq. Id. No. 2. In an aspect the living animal is a mouse or member of the mouse family.

In an aspect, a method for expressing a protein having a sequence shown in Seq. Id. No. 2 which comprises competently integrating a vector containing a gene comprising a polynucleotide having a sequence shown in Seq. Id. No. 1 into the genome of a living animal. In an aspect the living animal is a living mouse.

In an aspect, a genetically engineered expression vector comprises an expressing gene or part of sequence of an expressing gene comprising a polynucleotide having a sequence shown in Seq. Id. No. 1. In an aspect, the gene encodes a protein comprising a polypeptide having a sequence shown in Seq. Id. No. 2.

In an aspect, an engineered humanized antibody that binds to or reacts with a protein comprises a polypeptide having a sequence shown in Seq. Id. No. 2.

In an aspect, an oligo comprises an oligo synthesized according to the sequence of the gene comprising a polynucleotide having sequence shown in Seq. Id. No. 1. In an aspect, the oligo is double-stranded

In an aspect, an antisense oligo comprises an oligo based on a gene comprising a polynucleotide having a sequence shown in Seq. Id. No. 1. In an aspect, the oligo is single or double stranded.

In an aspect, a siRNA targeted to a gene comprises a polynucleotide having sequence shown in Seq. Id. No. 1.

In an aspect, a genetically engineered expression vector comprises an antisense oligo based on a polynucleotide having a sequence shown in Seq. Id. No. 1.

In an aspect, a method of down and up regulating a gene comprising a polynucleotide having a sequence shown in Seq. Id. No. 1 and its encoded protein comprising a polypeptide having a sequence shown in Seq. Id. No. 2 in control of tumor cell growth, invasion and metastasis.

In an aspect, a pharmaceutical composition is provided comprising an oligo comprising a part of sequence of the gene comprising a polynucleotide having sequence shown in Seq. Id. No. 1 with a suitable pharmaceutically acceptable carrier.

In an aspect, a pharmaceutical kit comprises a container housing an oligo having sequence of the gene comprising polynucleotide having sequence shown in Seq. Id. No. 1 with optionally a suitable pharmaceutical carrier.

A transgenic living mouse having competently integrated in its genome a functional genomic expressing comprising a gene comprising a polynucleotide having Seq. Id. No. 1. A transgenic living mouse having competently and capably integrated in it genome a functional genomic expression which expresses a protein having Seq. Id. No. 2.

In an aspect, a non-invasive method of using the expression products of a gene for drug discovery, said gene comprising a polynucleotide having a sequence shown in Seq. Id. No. 1 as an indicator of the effectiveness of administered candidate drug to a nonhuman living mammal having benign and malignant tumors and precancerous lesions and having competently integrated in its genome said gene, the method comprising applying sufficient heat to a tissue locus containing a suspected tumor in an amount and for amount of time and under conditions effective to activate the gene and forming a treated tissue locus, administering the candidate drug therapy to the mammal, obtaining a sample of the locus and analyzing the sample for the extent of presence of a protein comprising a polypeptide having a sequence shown in Seq. Id. No. 2 and determining the extent provides an indication of the therapy effectiveness of the administered drug, evaluating the effectiveness of the candidate drug on the cancer and making a prioritization of the development of the drug based on that effectiveness as a part of drug discovery.

In an aspect, a method to treat patients with benign and/or malignant tumors as well as precancerous lesions based on stress, including hyperthermia, inducible cell surface proteins.

In an aspect, a method to treat patients with benign and/or malignant tumors as well as precancerous lesions with stress, including hyperthermia, based tumor immunotargeting therapy.

### BRIEF DESCRIPTION OF THE DRAWINGS

FIG. 1 depicts a morphological comparison between NSY-CHR (chronic heat resistant, variant of NSY) cells maintained at 41° C. and NSY (Wild type) cells maintained at 37° C.

FIG. 2 depicts an agar gel of RT-PCR products of NSY-CHR and NSY cells (0.7% agar gel).

FIG. 3 depicts a northern blot of NSY-CHR and NSY cells.

FIG. 4 depicts HICSP expression on the surface of NSY and NSY-CHR cells (Immunofluorescence).

FIG. 5 depicts expression of HICSP in cell surface fractions precipitated with biotin-streptavidin technique from NSY and NSY-CHR cells.

FIG. 6 depicts expression of HICSP in whole cell lysates of NSY, NSY-CHS and NSY-CHR cells.

FIG. 7 depicts that expression of 270 kD and 130 kD heat-inducible cell surface proteins (HICSP) were significantly increased on the surface of NSY cells after heating at 41° C. for different period of time (analyzed by biotinylation and western blotting methods).

FIG. 8 depicts HICSP (270 kD) was induced and/or accumulated in tumor cells after heating at 41° C. (analyzed by western blotting in whole cell lysates).

FIG. 9 depicts expression of HICSP (270 KD) in human normal fibroblast after heating at 41° C. for indicated time (analyzed by western blotting in whole cell lysates). In this test, HICSP was undetectable.

FIG. 10 depicts that HICSP was increased on NSY cell surface after heating at 41° C. (hyperthermia) for 2 hour.

FIG. 11 depicts that HICSP was increased on TH29 human colon cancer cells after heating at 41 hC for 1 hour.

FIG. 12 depicts that expression of HICSP was increased on the surface of human breast cancer MCF7 cells after heating at 41° C. for 40 minutes.

FIG. 13 depicts that expression of HICSP protein on human fibroblast CRL7483 and malignant tumor CRL7484 cells maintained at 37° C. or heated at 41° C. for 1 hr.

FIG. 14 depicts that HICSP was induced and/or accumulated after challenging with lower heating temperature 40° C. for an indicated time in HT29 human colon adenocarcinoma cells.

FIG. 15A depicts that expression of HICSP was continuously increased after heating at 41° C. for 1 hr in HT29 human colon adenocarcinoma cells.

FIG. 15B depicts quantitative analysis of HICSP after heating at 41° C. for 1 hr.

FIG. 16 illustratively depicts tumor immunotargeting therapy with heat.

FIG. 17 is a cartoon showing prior art (tumor immunotargeting therapy without heat.

### DETAILED DESCRIPTION OF THE INVENTION

The inventor reports for the first time his isolation, purification (peptide) and characterization of HICSP protein having utility as an enhanced assay and therapeutic treatment for cancer and as a research tool. In an aspect the treatment comprises a hypertherrnia-based tumor immunotargeting diagnosis and therapy treatment regime treatment based on intentional hyperthermia and increased expression of HICSP protein. This discovery possesses credible, specific and substantial utility. The HICSP protein having Seq. Id No 2 is a target in diagnosis/treatment for benign and malignant tumors and precancerous lesions.

One aspect of this invention provides a functional method of diagnosis of the presence of benign and malignant tumors and precancerous lesions in a living mammal such as in a living human. The process involves providing a therapeutic antibody or binding portion thereof, probe, or ligand which binds to a portion of the protein. The protein itself is expressed by the intentional application of sufficient heat via hypothermia therapeutic means to a living human under conditions sufficient to induce the production of a protein serving as a biomarker wherein the presence of such protein is indicative of the presence of cancer in that living mammal.

More particularly a heat-inducible cell surface protein (HICSP) induced and/or accumulated by cell stress including stress from moderate hyperthermia (a temperature ranging from about 39° C. to about 42.5° C.) has been identified, characterized and isolated. HICSP (including a fragment, epitope thereof) is provocative i.e. it is functionally capable of triggering a mammalian immune defense system and producing antibodies in the presence of the protein. The HICSP protein and use of the characterizations thereof are extremely useful in that they provide a basis for novel methods of diagnosing and treating cancer and assessing the risk of cancer as herein described. Novel antibodies have been generated in connection therewith, which are useful as self-directed novel antibodies to use as guides to selectively diagnose, attack, target, locate and therapeutically treat cancer cells.

In a further aspect, the invention includes an isolated, purified and characterized gene comprising a gene sharing at least a 90% homology with a gene comprising a polynucleotide having a sequence shown in Seq. Id. No. 1. In an aspect, the invention includes an isolated protein comprising a protein sharing at least 90% homology with a protein comprising a polypeptide having a sequence shown in Seq. Id. No. 2.

In practice of this invention a gene which encodes and expresses the HICPS protein is deliberately effectively induced to express the HICPS protein above its background or normal expression level in a process which comprises applying finite heat to living cells, tissues, organs or a whole body for a time and in an amount effective and sufficient to cause activation of that gene with the resulting production of the HICPS protein. In an aspect the gene comprises a polynucleotide having a sequence shown in Seq. Id. No. 1 and the encoded protein (HICPS) comprises a polypeptide having a sequence shown in Seq. Id. No. 2.

Upon expression of the HICSP protein, one obtains a sample of the locus of that expressed protein, assays the sample for the presence of the expressed products of mRNA and protein of the gene and from that assay determines that the identified location of HICSP marks the location or locus of the tumor. The inventor has also discovered methods to tailor and customize drug therapy for cancer, methods to assess the risk to a mammal of bearing cancer and methods to assess the proliferation of a cancer in a mammal using this discovery.

As used herein, the term “peptide” includes any of a group of compounds comprising two or more amino acids linked by chemical bonding between their respective carboxyl and amino groups. The term “peptide” includes peptides and proteins that are of sufficient length and composition to affect a biological response, e.g. antibody production or cytokine activity whether or not the peptide is a hapten. Term “peptide” includes modified amino acids, such modifications including, but not limited to, phosphorylation, glycosylation, pegylation, lipidization and methylation.

As used herein, the term “polypeptide” includes any of a group of natural or synthetic polymers made up of amino acids chemically linked together such as peptides linked together. The term “polypeptide” includes peptide, translated nucleic acid and fragments thereof.

As used herein, the term “polynucleotide” includes nucleotide sequences and partial sequences, DNA, cDNA, RNA variant isoforms, splice variants, allelic variants and fragments thereof.

As used herein, the terms “protein”, “polypeptide” and “peptide” are used interchangeably herein when referring to a translated nucleic acid (e.g. a gene product). The term “polypeptide” includes proteins.

As used herein, the term “isolated polypeptide” includes a polypeptide essentially and substantially free from contaminating cellular components.

As used herein, the term “isolated protein” includes a protein that is essentially free from contamination cellular components normally associated with the protein in nature.

As used herein, the term “nucleic acid” refers to oligonucleotides or polynucleotides such as deoxyribonucleic acid (DNA) and ribonucleic acid (RNA) as well as analogs of either RNA or DNA, for example made from nucleotide analogs any of which are in single or double stranded form.

As used herein, the term “patient” and subject” are synonymous and are used interchangeably herein. In an aspect, the term “living animal” includes living mammals, including human and nonhuman living mammals such as mice, rodents, dogs and cats.

In an aspect the amount of expressed or translocated protein having Seq. Id. No. 2 is an effective amount, i.e. an amount which is effective to enable one to carry out this discovery.

As used herein, the term “expression” includes the biosynthesis of a product as an expression product from a gene such as the transcription of a structural gene into mRNA and the translation of mRNA into at least one peptide or at least one polypeptide.

As used herein, the term “sequencing” includes the process of identifying the order in which base pairs appear in DNA chains and identifying the order of amino acids in proteins. In sequencing researchers label copies of a DNA sequence with fluorescent markers, then run them through a sequencing machine. In proteins, amino acids are removed one at a time from the end of a protein and identified with an automated system. Sequencing methods for DNA chains and proteins are known in the art. An automated sequencing system is commercially available.

As used here, the terms “isoforms” and “splice variant” includes alternative occurring forms of RNA transcribed from a genome as well as polypeptides encoded by a splice variant of mRNA transcribed from a gene.

As used herein, the term “immunomodulator” includes such compounds as cytokines, stem cell growth factors, lymphotoxins, co-stimulatory molecules, hematopoietic factors and synthetic analogs of such molecules.

As used herein, the term “antibody fragment” is any useful portion of an antibody (Fab(s)), including an epitope, which binds the same antigen (protein comprising a polypeptide having a sequence shown in Seq. Id. No. 2) that is recognized and capably bound by an intact or nonfragmented antibody.

As used herein, the term “humanized antibody” and (“engineered human antibody”) includes recombinant proteins in which murine complementarity determining regions of a monoclonal antibody have been synthetically transferred or exchanged from heavy and light variable chains of the murine immunoglobulin into a human variable domain.

As used herein, the term “therapeutic agent” is any molecule or atom which is conjugated, fused or otherwise affixed to an antibody moiety to produce a conjugate which is useful for therapy.

As used herein, the term “label” includes a detectable label which includes any conjugatable molecule or atom to an antibody which can be conjugated to an antibody moiety to produce a detectable molecule useful for diagnosis and therapy. Useful nonlimiting labels include chelators, radioisotopes, radionuclides, fluorescent agents such as fluorescent proteins, and paramagnetic ions.

As used herein, the term “antibody” includes both an intact antibody, entire antibody, binding portions of an intact antibody, binding portions of a portion of an antibody, a useful antibody fragment or epitope. The term “antibody” also includes antigen binding forms of antibodies including any fragments comprising antigen binding forms and moieties. In an aspect the term “antibody” also refers to a polypeptide substantially encoded by an immunoglobulin gene or immunoglobulin genes, or fragments thereof which specifically bind and recognize an analyte (antigen).

As used herein, the phrase “specifically (or selectively) binds to an antibody” or specifically (or selectively) reactive with,“when referring to a protein, peptide, or polypeptide refers to a binding reaction that is determinative of the presence of the protein in a heterogeneous population of proteins and other biologics. Thus, under designated immunoassay conditions, specified antibodies bind to a particular protein and do not bind in a significant amount to other proteins which may be present in the sample.

As used herein, the term “immunoconjugate” includes a fused product or conjugate of an antibody component with a therapeutic agent or detectable label therewith.

As used herein, the term “fused antibody” means a recombinant molecule comprising an antibody component and a therapeutic agent. Useful nonlimiting therapeutic agents include immunomodulators and toxins.

As used herein, the term “tumor-associated antigen” is a protein normally not expressed, or if expressed then expressed at lower levels by a normal mammal cell. Of particular interest is a protein designated as HICPS (comprising a polypeptide having a sequence shown in Seq. Id. No. 2) having a molecular weight of about 270 and/or about 130K Dalton that is encoded and expressed on a tumor cell under stress conditions. In an aspect, the protein is expressed on the surface of a tumor cell.

As used herein, the term “mammal” includes living animals including humans and non-human animals such as murine, porcine, canine, rodentia and feline.

As used herein, the term “target protein” includes targets including an amino acid sequence expressed on a target cell such as on a tumor cell. In an aspect, the target protein is a protein having a sequence shown in Seq. Id. No. 2.

As used herein, the term “antisense” means a strand of RNA whose sequence of bases is complementary to messenger RNA.

As used herein, the term “siRNA” means short interfering RNA.

As used herein a “therapeutic amount” is an amount of antibody which produces a desired or detectable therapeutic effect on or in a mammal administered the antibody.

As used herein, the terms “heating and heated” include the application of an effective amount of heat or heat energy applied in a manner sufficiently directed to the mammal as a subject or target of the heat treatment. Useful methods for heating the mammal include the effective application of an effective microwave, radiowave, ultrasound and radiant heat and magnetic induction. Nonlimiting useful heat includes heat as applied by a heating blanket such as a thermostatically controlled device and heat as applied in a directed heating chamber.

As used herein the term “hyperthermia” comprises energy deposition to, in or on a target tissue and includes the application of pathogens (materials such as bacterial or viruses) into the body to effectively induce fever.

In an aspect, the term “hyperthermic” comprises the condition of heating being deliberately applied to a mammal in a manner, typically following a protocol, which deliberately raises mammal body temperature in a controlled desired manner above a normal nearly constant temperature. Organs of the human body, such as the brain, kidney, and heart, are maintained at a normal constant temperature of approximately 37.C. degree. Slight excursion of temperature or decrease of temperature have been noted in some individuals and are considered normal; however, the biophysical body control system of humans generally maintains the internal body temperatures at about 37 C. degrees. In an aspect the manner is stepwise, ramped up or a combination thereof.

As used herein the term “oligo” includes oligonucleotides which are polymers of nucleosides joined, generally, through phosphoester linkages.

As used herein, the terms “oligonucleotide” and “polynucleotide” are interchangeable and include single-stranded DNA (ssDNA), double-stranded DNA (dsDNA), single-stranded RNA (ssRNA) and double-stranded RNA (dsRNA), modified oligonucleotides and oligonucleosides and combinations thereof. The oligonucleotide can be linearly or circularly configured, or the oligonucleotide can contain both linear and circular segments.

As used herein, immunotherapy comprises use of antibodies as well as use of growth factors.

More in detail, in an aspect, the biological marker HICSP for tumor immunotargeting therapy is intentionally induced to be expressed/encoded and/or accumulated by stress including hyperthermia. Such inducing will produce an increased abnormal amount of expressed product (HICSP) protein. In that regard after reading the specification those of skill in the art will appreciate that abnormal expression levels or abnormal expression products (e.g., mutated transcripts, truncated or non-sense proteins) are identified by comparison to normal expression levels and normal expression products. Normal levels of expression or normal expression products can be determined for any particular population, subpopulation, or group of expressed products such as HICSP according to standard methods known to those of skill in the art.

A method and apparatus for performing selective intentional environmentally controlled and predetermined hyperthermia of a selected living mammal is carried out by placing that mammal in sufficient effective spatial proximity to a capably enabled functional Apparatus suitable and configurably enable to delivered an intentional controlled amount of energy to a mammal creating conditions effective to enable the carrying out of this discovery. In an aspect the apparatus is placed in sufficient effective spatial proximity to the mammal desired to be treated as a patient herein.

If desired the hyperthermia regime may be programmed into a computer equipped with a chip and software and communicative hardware to a hyperthermic apparatus and the mammal presented as a patient and recipient of the heat energy thereto. Once the desired or predetermined amount of heat energy has been capably delivered to the mammal, the unit is shut off and the mammal removed from spatial proximity to the apparatus or the apparatus removed from the spatial area of the mammal.

In an aspect, deliberately induced hyperthermia procedures include using a controlled systemic procedure for inducing hyperthermia to a living mammal.

A myriad of useful effective hyperthermic therapies can be used including one or more of whole body hyperthermia (WBH), superficial hyperthermia (SHT), loco-regional deep hyperthermia (DHT), intracavitary/peritoneal hyperthermic perfusion, intratumor hyperthermia (high frequency induced thermotherapy, intraperitoneal hyperthermic perfusion treatment, surface hyperthermia, intracavity hyperthermia, interstitial hyperthermia, prostatic thermotherapy and extracorporeal hyperthermia.

Typically however, hyperthermia is classified as local, regional, and whole-body hyperthermia (VVBH), all of which use external and internal heating devices and methods.

Living mammals typically self-control their core body temperature between narrow limits which enable them to be independent of other fluctuations in their surrounding non self-controllable environmental temperature. This is needed because enzymes are quite sensitive to changes in body core temperature. Typically those animals including mammals belong to a group called homeothermic, but at times it seems that most animals should be endotherms since they are able to maintain core body temperatures which remain remarkable constant. This discovery however involves a method and use of an apparatus which successfully produces an intentional controlled high body core mammalian safe temperature excursion for a time sufficient to carry out this discovery. Thereafter the energy input to the mammal inducing the intentional hyperthermia is shut off and the animals body core temperature returns to its normal operating range. This discovery is useful for such endotherm living mammals.

In this discovery the temperature excursion is such that the amount of temperature excursion is in the range from about 40° C. for 1 or 2 hrs to about 41° C. for 40 minutes or 1 hr and preferably from about 40° C. to about 41° C. above the mammal's statistically determinated core body temperature endothermic range.

Local hyperthermia refers to heat that is applied to a small area, such as a tumor. The area may be heated externally with high-frequency waves aimed at the tumor from a device outside the body. To achieve internal local heating, one of several types of sterile probes may be used, including thin, heated wires or hollow tubes filled with warm water; implanted microwave antennae and radiofrequency electrodes. Intratumoral hyperthermia is a local hyperthermia procedure for treating tumor diseases usually treatable by surgery. In an aspect, a special needle is inserted into the tumor and high frequency induced heat is applied to the local tumor. In an aspect, radiowaves are sent through small needles into a solid tumor.

Localized topical (i.e. superficial) hyperthermia includes heating cells with use of radiofrequency or microwave hyperthermal, infrared hyperthermia or ultrasound hyperthermia using ablation or lasers.

In such local hyperthermia applications, microwave, radio frequency and ultrasound may be used. In an aspect, microwave probes are placed in a mammalian body as the microwaves are applied and directed to solid tumors that range from about 1 cm to about 3 cm below the skin surface.

Generally a treatable tumor depth is in the range from an outer skin surface to an internal depth of about 8 cm below the outer skin surface. In an aspect, solid tumors are treated which are located in the chest wall, axilla, head, neck, breast, groin and tumors located in and beneath the mammalian skin. This includes melanoma and other skin cancers.

In regional hyperthermia, a mammalian organ or a limb is intentionally heated as a part of this discovery Magnets and devices that produce high energy are placed over the organ or limb to be heated. In another approach, termed extracorporal and referred to as hyperthermic perfusion, a selected portion (regional) of the living patient's blood is deliberately and controllably removed from the patient's body, heated and then pumped (perfused) into the region that is to be heated internally so that the patient's blood is heated. The warmed blood is returned to the patient's vascular system as part of the extracorporal blood heating system and process. Generally the extracorporal system is a continuously circulating system so that a steady state heating and circulation is maintained for patient comfort and survivability.

In whole body hyperthermia, hyperthermia is induced as a whole body systemic low temperature sustained hyperthermia such as in infrared saunas, infrared ozone sauna, fever therapy induced from biological injection such as BCG vaccine, Coley's toxins and mistle extract (viscum). However generally the hyperthermia treatment comprises high frequency radio waves directed to a living mammal which produces heat in a solid tumor in that mammal. In an aspect, during hyperthermia, extensive monitoring of the body internal and external temperatures is carried out using a three dimension deep heating phased array which is capable of imagining simultaneously with deep focused and regional heating to the mammal.

Generally intentional whole body hyperthermia (WBH) is used to heat the whole living mammalian body to a desired temperature which is held for minutes or further heated or held only in a selected temperature range for a short but sustained time. In such a WBH setting the mammalian patient is thermally isolated and infrared heat of different ranges of wavelengths is deposited on superficial tissues of the mammalian body until the desired temperature is achieved. During the application of hyperthermia typically the core body temperature is measured and carefully maintained.

Generally WBH is used in cases of metastatic cancer. Additional useful heating means include warm-water blankets, hot wax, inductive coils (such as those in electric blankets), or thermal chambers (similar to large incubators).

In a further aspect regarding WBH, medical doctors use the pyrogen Vacineurin or alpha-Interferon to stimulate fever. During such stimulation the mammal patient is carefully and constantly monitored by an intensive care unit complete with blood pressure, pulse and 3-channel ECG (electrocardiogram) In this aspect, the body temperature is raised moderately or extensively to the maximum by controlled deliberate exposure to infrared heat sourced in a special hyperthermia bed. The patient's head remains outside of the unit and is continuously cooled. Core body temperature, blood pressure, heart rhythm and oxygen saturation are monitored and strictly controlled in an intensive care unit.

In another aspect, WBH is induced in a mammal patient by infrared radiation by using a Medproducts unit (Medtronic World Headquarters, 710 Medtronic Parkway, Minneapolis, Minn. 55432-5604). The unit comprises a radiation unit on mobile stand, adjustable in height, whole body cabin, frame and insulated blankets with special head and eye protection for the mammal. In another aspect, a human body cabin is used having a stretcher or body support facility inside the unit. The human patient's temperature is raised by its absorbing radiant energy from the surface of the cabin.

Intentional controlled hyperthermia is advantageously used in large measure due to the recent development of multi-antenna applicators including their transforming networks and implementation of extensive dedicated systems for monitoring of E fields, e.g. electro optical sensors. Useful radiant heating devices for such exogenous increasing of mammal (patient) body-core temperatures including the enthermics Medical Systems and Aquatherm units which make use of a temperature (about 60° C.) hot cylinder around the mammal patients with 90% relative humidity of air. In an aspect, the cylinder emits long wave infrared-C radiation to the patient. A spectrum of long wave visible and adjacent infrared range (760-1400 mn) penetrate the skin layers.

Typical useful directed heating chambers for deliberately inducing controlled environment whole body hyperthermia include chambers for applying a finite amount of heat to a patient under carefully controlled conditions. An illustrative useful directed controlled heating chamber is manufactured and sold by Labthermics Technologies, Inc., 701 Devonshire Drive, Champaign, Ill. 61820, see http://www.labthermics.com. In an aspect, a useful microwave system for hyperthermia includes BSO Medical Systems, Salt Lake City, Utah, see http://www.bsdme.com. Such heating chambers are typically computer controlled with an operating computer, suitably equipped with functional software and capably instruction and communication enable to the heating chamber so as to provide an intentional induced heating under controlled conditions.

The intentional controlled hyperthermic treatment is generally carried out following a temperature-time protocol. Useful temperature time protocols include those where there is a constant temperature, time varied, patient varied and equipment varied hyperthermic heating of the living mammal as a function of time. If desired the protocol includes cycling of the application of heat, sequentially heating the mammal or staging the heating depending on the mammal's physiological response to hyperthermia treatment. The application of heat may be slow or rapidly ramped up.

In an aspect whole body hyperthermia is carried out along with immunotherapy. In an aspect the immunotherapy is carried out after the hyperthermia is complete or substantially complete. In an aspect the hyperthermia may be repeated and the immunotherapy may be repeated in a novel treatment regime.

In an aspect, a whole body intentional controlled pre-determined hyperthermia immunotherapy protocol comprises: 1) heating a mammalian patient at about 40° C. to about 41° C. for about 30 minutes to about 1 hr and then administrating conjugated antibodies against protein comprising polypeptides having Seq. Id. No. 2. 2) Repeating heat strategies: a) heating at 41° C. for at least 3 times with 24 hr interval, each time for an hour; b) heating at 41° C. at least for 3 times with 48 hr interval, each time for an hour; c)) heating at 41° C. at least for 3 times with unscheduled interval, each time for 30 minutes to an hour.

In an aspect, a non-invasive method of using a gene comprising a polynucleotide having a sequence shown in Seq. Id. No. 1 as a tumor locating agent in the treatment of cancer comprises:
applying heat to a tissue locus containing a suspected tumor in an amount and for amount of time effective to activate the gene and forming a treated tissue locus,
obtaining a sample of the locus by method of taking biopsy,
analyzing the sample for the presence of the products, mRNA and protein, of the gene comprising a polynucleotide having a sequence shown in Seq. Id. No. 1 and
determining that the presence marks the location or locus of the tumor.

In an aspect, a non-invasive method of using antibodies binding to a protein, comprising a polypeptide having as a sequence a sequence shown in Seq. Id. No. 2 as a tumor locating agent in the treatment of cancer comprises:
applying heat to a tissue locus containing a suspected benign and malignant tumor and precancerous lesion in an amount and for amount of time effective to induce expression of the protein forming a treated tissue locus,
obtaining a representative sample of the locus by a method of taking biopsy.
analyzing the sample for the presence of a protein comprising a polypeptide having a sequence shown in Seq. Id. No. 2 and
determining that the presence marks the location or locus of the tumor and
performing an imaging diagnosis by giving a subject conjugated antibodies with radioisotope. In an aspect the antibody binds to a protein comprising a polypeptide having a sequence shown in Seq. Id. No. 2.

In another aspect, an effective amount of conjugated antibodies against protein comprising polypeptides having Seq. Id. No. 2 can be administrated immediately before and after hyperthermia, or the same time as hypothermia.

Advantageously from a time view point the biological marker HICSP for tumor immunotargeting therapy can be induced and/or accumulated by such hyperthermic heating at 40° C. to 41° C. for as little as about 30 minutes. Once the HICSP biological marker is induced and/or accumulated it can be maintained on cell surface for at least 1 hour and continuously increased after heating.

A temperature of 41° C. is homogenously achievable on a solid tumor on body surface in a clinic. 41° C. is also achievable to the tumor in a deep site of the body by heating probe (interstitial therapy) in clinic. Furthermore whole body hyperthermia (fever range) is therapeutically tolerable (i.e. survivable to that patient without lingering injury) and in that situation the novel hyperthermia-based tumor immunotargeting therapy herein is also suitable for the patients with blood malignant tumors and metastasis.

In an aspect this discovery, the biological marker HICSP for tumor immunotargeting therapy is deliberately manipulated by different time and heating protocols such as repeatedly heating to maximum HICSP expression in patients with cancer that do not increase the expression of HICSP by initial heating at 41° C. for about 30 minute or one hour.

Applicant has discovered the HICSP biomarker and its use for noninvasively diagnosing a living mammal for cancer to determine whether cancer has localized and if so, to determining that localization. This is an important advantage of this discovery in that the following therapy can be site directed to that localization thus saving valuable diagnostic and therapy time and hopefully saving that patient's life.

In that regard a method of using the HICSP as marker in diagnosing a mammal for the presence of cancer is provided in a process which comprises activating a gene comprising a polynucleotide having a Seq. Id. No. 1 in a cell thereof or inducing the gene to encoded protein comprising a polypeptide having a sequence shown in Seq. Id. No. 2 within a tissue locus which comprises treating a suspect tumor containing tissue locus in a hyperthermic manner effective to induce activation of the gene or its encoding of the protein, determining if the protein is present in a sample from the tissue and if the protein is detectably presented, determining that cancer is likely present in the tissue locus.

In another aspect, a method of treating benign and malignant tumors comprises administering an effective amount of antibodies against a protein comprising polypeptides having a sequence shown in Seq. Id. No. 2 on tumor or target cells to block signal transduction pathways which control at least one of cell proliferation, tumor cell invasion and metastasization.

In another aspect, a method of diagnosing a mammal for the presence of cancer comprises activating a gene comprising a polynucleotide having a Seq. Id. No. 1 in a cell thereof or inducing the gene to encoded protein comprising a polypeptide having a sequence shown in Seq. Id. No. 2 within a tissue locus which comprises treating a suspect tumor containing tissue locus in a hyperthermic manner effective to induce activation of the gene or its encoding of the protein, determining if the protein is present in a sample from the tissue and if the protein is detectably presented, determining that cancer is likely present in the tissue locus.

In another aspect, a method of diagnosing cancer by detecting or inducing the expression of a protein comprising a polypeptide having a sequence shown in Seq. Id. No. 2 from a cell within a tissue locus which comprises treating a suspect tumor containing tissue locus in a hyperthermic manner sufficient to induce expression of a protein having a sequence shown in Seq. Id. No. 2 determining if the protein is present in a sample from the tissue and if the protein is detectably present, determining that cancer is likely present in the tissue locus.

In another aspect, a method of diagnosing cancer by detecting a gene comprising a polynucleotide having a sequence shown in Seq. Id. No. 1 in the cells from body fluids, such as scrum tears, sweat, urine, gastric and intestinal fluids, as well as saliva, various mucous discharges, and sinovial fluids.

In another aspect, a method of diagnosing cancer by detecting or quantitating mRNA of a gene comprising a polynucleotide having a sequence shown in Seq. Id. No. 1 in the cells from body fluids, such as scrum tears, sweat, urine, gastric and intestinal fluids, as well as saliva, various mucous discharges, and sinovial fluids.

In another aspect, a method of diagnosing cancer by detecting or quantitating a protein comprising a polypeptide having a sequence shown in Seq. Id. No. 2 from body fluids, such as scrum tears, sweat, urine, gastric and intestinal fluids, as well as saliva, various mucous discharges, and sinovial fluids.

In another aspect, a method of therapeutically effectively treating a mammal having a tumor comprises hyperthermically treating a suspect tumor locus in the mammal sufficient to activate a gene comprising a polynucleotide having a sequence shown in Seq. Id. No. 1 in a cell or inducing the gene encoded protein comprising a polypeptide having a sequence shown in Seq. Id. No. 2, analyzing for and determining the presence of the protein and determining whether to apply anti-tumor therapy to tissue based on that presence.

In an aspect, a method for effectively treating a living mammal comprising administering an effective amount of an anti-tumor agent or a functional derivative thereof, alone or in combination with a tumor-specific antibody or other tumor-directing agent to the mammal wherein the antibody recognizes a protein comprising a polypeptide having a sequence shown in Seq. Id. No. 2.

In another aspect, a method of effectively medically treating a living mammal comprises administering an effective amount of a toxic tumor therapy to a tumor locus of the mammal, comprising administering a therapeutically effective amount of an anti-cancer agent, wherein the agent is guided to the tumor locus by an antibody target comprising a protein comprising a polypeptide having a sequence shown in Seq. Id. No. 2 in the locus.

In an aspect, an isolated and characterized antibody-antigen system comprises a protein having as a sequence a sequence shown in Seq. Id. No. 2 and an antibody capably recognizing that protein.

In an aspect, an isolated and characterized antibody composition comprises an antibody binding to a protein comprising a polypeptide having sequence shown in Seq. Id. No. 2 and a suitable carrier.

The antibodies of this discovery comprise immune system related proteins with each antibody comprising four polypeptides having two heavy chains and two light chains to form a “Y” shaped molecule. It is understood that there may be several Fabs which comprise the novel antibodies of this discovery and that such Fabs are likewise covered under the claims of this patent application. It is further understood however that the description provided herein including the extensive functionality of the antibody is sufficient to make it clear to those of skill in the art the antibodies included in this discovery even with variations in Fabs.

In an aspect, a method of reducing at least one of the size and number of tumor cells in a living mammal comprises effectively administering to the mammal an effective amount of an antibody that binds to a protein comprising a polypeptide having as a sequence a sequence shown in Seq. Id. No. 2. In an aspect, the antibody is coupled to a tumor cytotoxic agent. In an aspect the administration is carried out such that the antibody is provided to the living mammal in a manner and condition sufficient for binding to a tumor in a localized area of the living mammal.

In an aspect, a method of imaging a tumor in a mammal comprises administering to the mammal a tumor-imaging amount of a detectably labeled antibody binding to a protein comprising a polypeptide having a sequence shown in Seq. Id. No. 2.

In an aspect, a method of preventing, treating, diagnosing, grading, prognosing or ameliorating a tumorous medical condition in a living mammal comprises administering to the mammal a therapeutically effective amount of a provocative antibody having sufficient specific binding capability to a protein comprising a polypeptide having as a sequence a sequence shown in Seq. Id. No. 2. In an aspect, the administration is in situ. In an aspect, the administration is external.

In an aspect, a method of diagnosing a pathological condition or susceptibility to a pathological condition in a living mammal comprises administering a hyperthermic treatment to the mammal sufficient to induce detectable production of a protein comprising a polypeptide having a sequence shown in Seq. Id. No. 2 over a normal production amount of the protein, determining the extent of increased production and determining susceptibility based on the production.

In an aspect, immunological binding assays (described hereinafter) are employed to assay for the presence of HICSP as an adjunctive assay relating to effective safe hyperthermia and in the production of (self directed) antibodies which recognize HICPS60.

As noted above, immunotherapy can be administered following intentional controlled safe therapeutic hyperthermia. In an aspect as part of immunotherapy novel monoclonal antibodies and polyclonal targeted to cell surface novel protein comprising a polypeptide having a sequence shown in Seq. Id. No. 2 are administered to a mammal as a selective carrier of toxins, chemotherapeutic agents or radionuclides employed to diagnose and/or treat cancer.

In an in vivo approach, antibodies are administered to a patient by administering an immunotherapeutic composition as a pharmaceutical composition to the mammal. Typically the administration is carried out in a therapeutically effective manner and amount to a subject that has a tumor. Such an in vivo administration can provide at least one beneficial physiological effect selected from the group consisting of decreased number of tumor cells, decreased metastasis, decreased size of one or more solid tumors, increased necrosis of a tumor, decreased rate of spread of the tumor.

In an aspect, monoclonal and polyclonal antibodies are prepared that react with i.e. recognize a protein comprising a polypeptide having Seq. Id. No. 2 on cancer cells in accordance with accepted laboratory practice. (A useful Monocolonal antibody production is described in Monoclonal Antibody Production, A Report of the Committee on Methods of Producing Monoclonal Antibodies Institute for Laboratory Animal Research National Research council, National Academy Press, Washington D.C. 1999 which is incorporated herein by reference in its entirety.) Monoclonal antibody therapy is a passive immunotherapy as the antibodies are produced in a laboratory rather than by living mammal immune system itself.

In an aspect, monoclonal antibodies are produced in a mouse which is immunized by injection of an antigen (e.g. a protein comprising a polypeptide having Seq. Id. No. 2) to stimulate the production of antibodies targeted against that antigen in the mouse. (see Kohier & Milstein, Eur. J Immunol. 6:511-519 (1976)).

In an aspect, the antibody forming cells are isolated (i.e. harvested) from the mouse's spleen. Monoclonal antibodies (hybridomas) are produced by fusing the single antibody-forming cells to inhibit tumor (cancer) cells grown in culture.

Alternative methods of immortalization include transformation with Epstein Barr Virus, oncogenes, or retroviruses, or other methods well known in the art. In an aspect, colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., Science 246:1275-1281 (1989).

After reading this specification those of skill in the art will recognize methods of producing polyclonal and monoclonal antibodies that can react specifically with HICSP (see, e.g., Coligan, Current Protocols in Immunology (1991); Harlow & Lane, supra; Goding, Monoclonal Antibodies: Principles and Practice (2d ed. 1986); and Kohier & Milstein, Nature, 256:495-497 (1975) and (see, e.g, Huse et al., Science 246:1275-1281 (1989); Ward et al., Nature 341:544-546 (1989)). Monoclonal antibody production may be effected by techniques which are well-known in the art. Basically, the process involves first obtaining immune cells (lymphocytes) from the spleen of a mammal (e.g., mouse) which has been previously immunized with the antigen of interest either in vivo or in vitro. The antibody-secreting lymphocytes are then fused with (mouse) myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature 256:495 (1975), which is hereby incorporated by reference.

Mammalian lymphocytes are immunized by in vivo immunization of the animal (e.g., a mouse) with the protein or polypeptide of the present invention. Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by standard and well-known techniques, for example, by using polyethylene glycol (“PEG”) or other fusing agents (See Milstein and Kohler, Eur. J. Immunol. 6:511 (1976), which is hereby incorporated by reference). This immortal cell line, which is preferably murine, but may also be derived from cells of other mammalian species, including but not limited to rats and humans, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies. Such antibodies can be raised by administering the protein or polypeptide of the present invention subcutaneously to New Zealand white rabbits which have first been bled to obtain pre-immune serum. The antigens can be injected at a total volume of 100 .mu.l per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits 20 are euthenized with pentobarbital 150 mg/Kg IV. This and other procedures for raising polyclonal antibodies are disclosed in E. Harlow, et. al., editors, Antibodies: A Laboratory Manual (1988), which is hereby incorporated by reference.

In an aspect an immunogen is used to produce antibodies that specifically react and recognize with HICSP. For example, recombinant HICSP or a antigenic fragment thereof such as the core or tail domain, is isolated. Using the sequence listing herein, a recombinant protein can be expressed in eukaryotic or prokaryotic cells, and purified using a vector and a host cell. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the HICSP protein.

More in detail, methods generally producing polyclonal antibodies are known to those of skill in the art. An inbred strain of mice or rabbits is immunized with the protein using a standard adjuvant, such as Freund's adjuvant, and a standard immunization protocol. The animal's immune response to the immunogen preparation is monitored by taking test bleeds and determining the titer of reactivity to HICPS. When appropriately high titers of antibody to the immunogen are obtained, blood is collected from the animal and antisera are prepared. Further fractionation of the antisera to enrich for antibodies reactive to the protein can be done if desired (see Harlow & Lane, supra). In as aspect, monoclonal antibodies and polyclonal sera are collected and tittered against the immunogen protein HICSP in an immunoassay, for example, a solid phase immunoassay with the immunogen HICSP immobilized on a solid support. Polyclonal antisera with a titer of 10⁴ or greater are selected and tested for their cross reactivity against non-HICSP protein or even other homologous proteins from other organisms, using a competitive binding immunoassay. Specific polyclonal antisera and monoclonal antibodies will usually bind with a K_{D} of at least about 0.1 mM, more usually at least about 1 μM, preferably at least about 0.1 μM or better, and most preferably, 0.01 μM or better.

For a review of immunological and immunoassay procedures, see Basic and Clinical Immunology (Stites & Terr eds., 7th ed. 1991). Moreover, the immunoassays of the present invention can be performed in any of several configurations, which are reviewed extensively in Enzyme Immunoassay (Maggio, ed., 1980); and Harlow & Lane, supra.

In an aspect, immunological binding assays are employed to assay for the presence of HICSP. In an aspect such assays are employed to determine the presence of HICSP during and after hyperthermia.

In an embodiment, HICSP is detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology Volume 37: Antibodies in Cell Biology (Asai, ed. 1993); Basic and Clinical Immunology (Stites & Terr, eds., 7th ed. 1991). Immunological binding assays (or immunoassays) typically utilize a “capture agent” to specifically bind to and often immobilize the analyte (in this case the HICPS or antigenic subsequence thereof). The capture agent is a moiety that specifically binds to the analyte. The antibody (anti-HICSP) may be produced by any of a number of means well known to those of skill in the art and as described above.

Throughout the assays, incubation and/or washing steps may be required after each combination of reagents. Incubation steps can vary from about 5 seconds to about several hours, preferably from about 5 minutes to about 24 hours. However, the incubation time will depend upon the assay format, analyte, volume of solution, concentrations, and the like. Usually, the assays will be carried out at ambient temperature, although they can be conducted over a range of temperatures, such as 10° C. to 40° C.

In competitive assays, the amount of HICSP (analyte) present in the sample is measured indirectly by measuring the amount of an added (exogenous) analyte (i.e., the HICSP) displaced (or competed away) from a capture agent (anti-HICSP antibody) by the analyte present in the sample. In one competitive assay, a known amount of, in this case, the HICSP is added to the sample and the sample is then contacted with a capture agent, in this case an antibody that specifically binds to the HICSP. The amount of HICSP bound to the antibody is inversely proportional to the concentration of HICSP present in the sample. In a particularly preferred embodiment, the antibody is immobilized on a solid substrate. The amount of the HICSP bound to the antibody may be determined either by measuring the amount of HICSP present in a HICSP/antibody complex, or alternatively by measuring the amount of remaining uncomplexed protein. The amount of HICSP may be detected by providing a labeled HICSP molecule.

In an aspect, the immunoabsorbed and pooled antisera are then used in a competitive binding immunoassay as described above to compare a second protein, thought to be perhaps the protein of this invention, to the immunogen protein (i.e., HICSP having a sequence shown in Seq. Id. No. 2). In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the protein partially encoded by Seq. Id. No. 2 that is required to inhibit 50% of binding, then the second protein is said to specifically bind to the polyclonal antibodies generated to a HICSP immunogen.

In an aspect, a kit contains primary antibody, secondary antibody that conjugated with detectable markers, like fluorescence (FITC etc.) and alkaline phosphatase. The secondary antibody is the antibody against primary antibody. Block buffer 10% serum of the species generated secondary antibody in PBS (phosphate buffered saline) or 10% BSA (bovine serum albumin). In this case primary antibody is the rabbit antibody against HICSP protein and secondary antibody should be goat, donkey etc anti-rabbit immuno-globulin. The blocking buffer should be 10% goat or donkey serum in PBS.

In an aspect, a kit contains primary antibody only, no secondary antibody that conjugated with detectable markers, like fluorescence (FITC etc.) and Alkaline phosphatase in it. Instead the primary antibody was conjugated with detective markers such as fluorescence (FITC etc) Block buffer is 10% BSA (bovine serum albumin) or 10% serum of the species from which antibody was generated. In this case primary antibody is the rabbit antibody against HICSP protein. The blocking buffer should be 10% BSA (bovine serum albumin) or rabbit serum or donkey serum in PBS.

It is often desirable to minimize non-specific binding in immunoassays, particularly, where the assay involves an antigen or antibody immobilized on a solid substrate it is desirable to minimize the amount of nonspecific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

In an aspect, a procedure of transfecting a gene comprising polynucleotide having Seq. Id No. 1. is hereinafter in detail:

The day before transfection, culture cells in dish; Dilute suitable amount DNA (how much amount depending on what culture format to be used, like 96 well plate, 48 well plate etc.) dissolved in TE buffer with DNA-condensation buffer to a suitable amount; Incubate at room temperature (15° C.-25° C.) for about 2to about 5 minutes then spin down the mixture for a few seconds to remove drops from the top of the tube; Add suitable amount of Effectan Transfection Reagent to the DNA-Enhancer mixture; Qiagen, 27220 Tumberry Lane, Suite 200, Valencia, Calif. 91355, USA.

Incubate the samples for 5-10 minutes at room temperature to allow transfection-complex formation. While the complex formation take place, gently aspirate the grow medium from the plate, and wash cells once with suitable amount of Phosphate Buffered Saline (PBS).

Then add suitable amount of fresh growth medium. Add suitable amount medium to the tube containing the transfection complexes; After mixing, add the transfection complex to the tissue culture dish. Incubate the cells with the transfection complexes under their normal growth conditions for an appropriate time for expression of the transfected gene. The incubation time is determined by the assay and gene used; For transient transfection, assay cells for expression of the transfected gene; For stable transfection, passage cells 1:5 to 1:10 into the appropriate selective medium about 24-48 hours after transfection. Maintain cells in selective medium until colonies appear.

In an aspect, a monoclonal antibody is prepared and used as a specific probe to track down and purify the specific antigen (protein) that induced its formation thereby pinpointing the locus of the cancer. In an aspect, corresponding polyclonal antibodies are generated utilizing a sequence of amino acid as immunogens. Polyclonal antibodies are characterized as antibodies having multiple binding sites.

In an aspect, naked monoclonal, naked polyclonal, conjugated monoclonal and polyclonal antibodies are useful in cancer treatments in this invention.

Without being bound by theory it is believed that the naked monoclonal antibodies and naked polyclonal antibodies attach themselves to the novel protein (comprising a polypeptide having a sequence shown in Seq. Id. No. 1) on the surface of cancer cells. In an aspect, the novel naked monoclonal antibodies are “said” to be associated with this novel protein (HICSP).

Conjugated monoclonal and conjugated polyclonal antibodies are especially useful herein in that they can be used for treating cancer by administering a lethal effective amount of anti-cancer agent or radiolabeled antibody comprising a conjugated antibody binding to a tissue locus presenting as a target thereto a protein comprising a polypeptide having a sequence shown in Seq. Id. No. 2 in a tissue locus.

Conjugated monoclonal antibodies are those that are joined to a chemotherapy drug, radioactive particle, or a toxin (a substance that poisons cells). In an aspect, the toxin is a cancer cytotoxin.

As used herein, “conjugated monoclonal antibodies” are those antibodies that are individually joined to drugs, toxins, or radioactive atoms, and used as delivery vehicles to transport drugs, toxins, or radioactive atoms to the cancer cells via the mammalian vascular system. Conjugated antibodies MAbs are also sometimes referred to as conjugated Mabs, and “tagged,” “labeled,” or “loaded.” MAbs with chemotherapy drugs attached are generally referred to as chemolabeled.

As to the latter, useful drugs cytotoxic to cancer which comprise a portion of a monoclonal and polyclonal conjugated antibody (chemolabeled antibody) include aldesleukin, alemtuzumab, alitretinoin, allopurinol, altretamine, amifostine, amifostine, anastrozole, anastrozole, arsenic trioxide, BCG Live, bexarotene, bleomycin, calusterone, capecitabine, carboplatin, carmustine, celecoxib, chlorambucil, cisplatin, cladribine, cyclophosphamide, cytarabine, dacarbazine, cdactinomycin, carbepoetin alfa, daunorubicin liposomal, denileukin diftitox, dexrazoxane, docetaxel, doxorubicin, dromostanolone, Elliot's B solution, epirubicin, epoetin alfa, estramustine, etoposide phosphate, exemestane, Filgrastim, floxuridine, fludarabine, fulvestrant, gemcitabine, gemtuzumab, gosereling acetate, hydroxyurea, Ibritumomab Tiuxetan, idarubicin , ifosfamide, imatinig mesylate, Interferon alfa-2a, Interferon alfa-2b, irinotecan, letrozole, leucovorin, levamisole, mechlorethamine, megestrol acetate, melphalan, L-PAM, mercaptopurine 6-MP, mesna, methotrexate, methoxsalen, mitomycin C, mitotane, mitoxantrone, Androlone phenpropionate, Nefetumomab, Oprelvekin, oxaliplatin, paclitaxel, pamidronate, pegademase, Pegaspargase, pegfilgrastim, entostatin, pipobroman, plicamycin mithramycin, porfimer sodium, procarbazine, quinacrine, Rasburicase, Rituximab, Sargramostim, streptozocin, talc, tamoxifen, Trastuzumab, tretinoin ATRA, uracil mustard, valrubicin, vinblastine, tamoxifen, temozolomide, teniposide VM-26, testolactone, thioguanine 6-TG, thiotepa, topotecan, toremifene, Tositumomab, vincristine, vinorelbine and zoledronate.

MAbs with radioactive particles attached are referred to as radiolabeled, and this type of therapy is known as radioimmunotherapy (“RIT”). Mabs may be employed to detect, diagnose and treat cancer herein as an immunotherapy. Such radiolabeled antibodies can be used to detect areas of cancer spread in the body as functional radionuclides.

A tumor may be imaged or treated in a mammal by a process which comprises administering to the mammal a tumor imaging amount or tumor toxic amount of a detectably radiolabeled antibody binding to a protein comprising a polypeptide having a sequence shown in Seq. Id. No. 2.

A tumor may be treated in a mammal by a process which comprises administering to the mammal a tumor toxic amount of antibody conjugated with nanoparticles that contain therapeutic reagent or temperature sensitive liposome particles which contain therapeutic reagent. The conjugated antibodies bind to a protein comprising a polypeptide having a sequence shown in Seq. Id. No. 2.

Temperature sensitive liposome particles are sensitive to moderate hyperthermia (ranging from 39° C. to 42.5° C.). Therapeutic reagents will be released from the particles by continuously heating or repeat heating after initial heating and administering the conjugated antibody.

The radiolabels may be incorporated into the antibodies by any of a number of means well known to those of skill in the art. However, in a preferred embodiment, the label is simultaneously incorporated during the amplification step in the preparation of the nucleic acids. Thus, for example, polymerase chain reaction (“PCR”) with labeled primers or labeled nucleotides will provide a labeled amplification product. In another preferred embodiment, transcription amplification using a labeled nucleotide (e.g., fluorescein-labeled UTP and/or CTP) incorporates a label into the transcribed nucleic acids.

Alternatively, a label may be added directly to an original nucleic acid sample (e.g., mRNA, poly A⁺ mRNA, cDNA, etc.) or to the amplification product after the amplification is completed. Means of attaching labels to nucleic acids are well known to those of skill in the art and include, for example, nick translation or end-labeling (e.g., with a labeled RNA) by phosphorylation of the nucleic acid and subsequent attachment (ligation) of a nucleic acid linker joining the sample nucleic acid to a label (e.g., a fluorophore).

The particular label or detectable group used in the assay is not a critical aspect of the invention, as long as it does not significantly interfere with the specific binding of the antibody used in the assay.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound.

The molecules can also be conjugated directly to signal generating compounds, e.g., by conjugation with an enzyme or fluorophore.

Detectable labels suitable for use in the present invention include any composition detectable by spectroscopic, radioisotopic, photochemical, biochemical, immunochemical, electrical, optical or chemical means. Useful labels in the present invention include biotin for staining with labeled streptavidin conjugate, magnetic beads, fluorescent dyes (e.g., fluorescein, texas red, rhodamine, green fluorescent protein, and the like), radiolabels (e.g., ³H, ¹²⁵I ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241 all of which are incorporated herein in their respective entirety by reference.

Tritium labeling procedures are described in U.S. Pat. No. 4,302,438, which is hereby incorporated by reference. Iodinating, tritium labeling, and .sup.35 S labeling procedures especially adapted for murine monoclonal antibodies are described by Goding, J. W. (supra, pp 124-126) and the references cited therein, which are hereby incorporated by reference Other procedures for iodinating biological agents, such as antibodies, binding portions thereof, probes, or ligands, are described by Hunter and Greenwood, Nature 144:945 (1962), David et al., Biochemistry 13:1014-1021 (1974), and U.S. Pat. Nos. 3,867,517 and 4,376,110, which are hereby incorporated by reference. Radiolabeling elements which are useful in imaging include .sup.123 I, .sup.131 I, .sup.111 In, and .sup.99m Tc, for example. Procedures for iodinating biological agents are described by Greenwood, F. et al., Biochem. J. 89:114-123 (1963); Marchalonis, J., Biochem. J. 113:299-305 (1969); and Morrison, M. et al., Immunochemistry, 289-297 (1971), which are hereby incorporated by reference. Procedures for .sup.99m Tc-labeling are described by Rhodes, B. et al. in Burchiel, S. et al. (eds.), Tumor Imaging: The Radioimmunochemical Detection of Cancer, New York: Masson 111-123 (1982) and the references cited therein, which are hereby incorporated by reference. Procedures suitable for sup.111 In-labeling biological agents are described by Hnatowich, D. J. et al., J. Immul. Methods, 65:147-157 (1983), Hnatowich, D. et al., J. Applied Radiation, 35:554-557 (1984), and Buckley, R. G. et al., F.E.B.S. 166:202-204 (1984), which are hereby incorporated by reference.

In the case of a radiolabeled biological agent, the biological agent is administered to the patient, is localized to the tumor bearing the antigen with which the biological agent reacts, and is detected or “imaged” in vivo using known techniques such as radionuclear scanning using e.g., a gamma camera or emission tomography. See e.g., A. R. Bradwell et al., “Developments in Antibody Imaging”, Monoclonal Antibodies for Cancer Detection and Therapy, R. W. Baldwin et al., (eds.), pp. 65-85 (Academic Press 1985), which is hereby incorporated by reference. Alternatively, a positron emission transaxial tomography scanner, such as designated Pet VI located at Brookhaven National Laboratory, can be used where the radiolabel emits positrons (e.g., .sup. 11 C, sup. 18 F, sup. 15O, and sup.13 N).

Means of detecting such labels are well known to those of skill in the art. Thus, for example, radiolabels may be detected using photographic film or scintillation counters; fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

Any metallic radioisotope capable of being detected in a diagnostic procedure can be employed to prepare a functional radionuclide.

Any metallic radioisotope capable of being detected in a PET or SPECT diagnostic imagining procedure can be employed as a functional radionuclide. Suitable nonlimiting examples of useful radionuclides include: Actinium-₂₂₅, Astatine-₂₁₁, Bismuth-₂₁₂, Bismuth-₂₁₃, Bromine-₇₅, Bromine-₇₆, Carbon-₁₁, Cerium-₁₄₁, Chromium-₅₁, Copper-₆₀, Copper-₆₁, Copper-₆₂, Copper-₆₄, Copper-₆₇, Dysprosium-₁₆₆, Fluorine-₁₈, Gadolinium-₁₅₂, Gadolinium-₁₅₃, Gold-₁₉₅m, Holmium-₁₆₆, Indium-₁₁₁, Indium-₁₁₃ₘ, Iodine-₁₂₃, Iodine-₁₂₄, Iodine-₁₂₁, Iron-₅₅, Iron-₅₉, Lutetium-₁₇₇, Nitrogen-₁₃, Oxygen-₁₅, Palladium-₁₀₃, Radium-₂₂₃, Radium-₂₂₄, Rhenium-₁₈₆, Rhenium-₁₈₈, Rubidium-₈₁, Rubidium-₈₂, Rubidium-₈₆, Ruthenium-₁₀₃, Ruthenium-₁₀₆, Samarium-₁₅₃, Scandium-₄₆, Tantalum-₁₇₈, Technetium-₉₄ₘ, Technetium-₉₉ₘ, Thallium-₂₀₁, Titanium-₄₅, Ytterbium-₁₆₉, Yttrium-₈₆, Yttrium-₉₀, and Zirconium-₈₉. In an aspect technetium-99m is used for SPECT imaging studies, and rhenium-188, rhenium-186, copper-64 and yitrium-90 are useful for radiotherapy of breast tumors.

A useful text on PET is clinical positive emission tomography, Gustav K. Schulthess, Lipcott, Williams & Williams 2000.

In an aspect, the method of Immunofluorescence staining, Elisa (Enzyme-Linked immunosorbent assay), Immunoautoradiography and western blot etc. will be used for detection and quantitation of a protein comprising a polypeptide having a sequence shown in Seq. Id. No. 2. Useful citations: Methods in Cell Biology: Flow Cytometry, Part A by Zbigniew Darzynkiewicz (Editor), et al 1994; Microscopy, Immunohistochemistry, and Antigen Retrieval Methods For Light and Electron Microscopy by M. A. Hayat, 2002; Methods in Cell Biology: Flow Cytometry, Part B by Zbigniew Darzynkiewicz (Editor), et al 1994; Immunoenzyme Multiple Staining Methods (Microscopy Handbook, 45) by C. M. Van Der Loos, et al 2000; Manual of immunological Methods by P. Brousseau, et al 1998.

In an aspect, the antibody HICSP is effectively incorporated in a pharmaceutic composition suitable for administration to a living mammal. One or more antibodies may be so incorporated. Useful antibodies include naked and conjugated monoclonal and polyclonal conjugated antibodies.

In an aspect, antibody compositions comprising naked monoclonal and polyclonal antibodies and conjugated monoclonal and polyclonal antibodies comprising chemotherapeutic compounds, toxic agents and radioligands are employed in the form of pharmaceutical preparations.

In an aspect, such pharmaceutical preparations are made in a manner well known in the pharmaceutical art. In an aspect, one preparation utilizes a vehicle of physiological saline solution comprising at least one of a chemotherapeutic compound, toxic agent and radioligand is combined with a pharmaceutically acceptable carrier. It may also be desirable that a suitable buffer be present in the composition which may include sterile water.

In an aspect, the carrier can also contain other pharmaceutically-acceptable excipients and additives for modifying or maintaining pH, osmolarity, viscosity, clarity, color, sterility, stability, rate of dissolution, or odor of the formulation. Similarly, the carrier may contain still other pharmaceutically acceptable excipients for modifying or maintaining release or absorption or penetration.

It is also contemplated that some formulations are more conveniently administered orally. Such formulations are preferably encapsulated and formulated with suitable carriers in solid dosage forms.

In an aspect a conjugated antibody comprising a radiolabel portion along with a cancer cytotoxic agent is employed in a pharmaceutical composition administered to a living mammal. In an aspect the immunotherapy comprises administration of two or more antibodies as part of an immuntherapy regime. Radiolabeled antibodies are employed to diagnose the presence of a tumor, the location of a tumor and to deliver a lethal amount of radiation to that tumor. Immuntoxins are also employed in conjugated antibodies.

The effective amount of such antibody administered must be determined empirically.

Parenteral routes of administration to mammals of such pharmaceutical compositions include but are not limited to electrical (iontophoresis) or direct injection such as direct injection into a central venous line, intravenous, intramuscular, intraperitoneal, intradermal, or subcutaneous injection. Compositions suitable for parenteral administration include, but are not limited, to pharmaceutically acceptable sterile isotonic solutions.

Diagnosis and treatment are important aspect of this invention. In an aspect, Positron Emission Tomography, (PET) and SPECT and microPET are useful radioimaging diagnostic imaging standard medical procedures that during data acquisition produce (i.e. capture and optionally record) images of the body's biological functions and in an aspect, are used to determine the extent of malignant disease as part of the hyperthermia immunotherapy regime. In an aspect, these imagining procedures show the presence and distribution of a radiolabeled detectable functionally emitting radiolabeled chemical i.e. a radionuclide that is also referred to as PET or SPECT or microPET radioligand. Advantageously, these imaging procedures depict metabolic characteristics of tissues.

MicroPet® is also useful in this diagnostic imaging using this discovery. MicroPET® is a dedicated PET scanner designed for high resolution radio imaging of small laboratory animals. One such scanner is available from Concorde Microsystems, Inc. 10427 Cogdill Rd, Suite 500 Knoxville, Tenn. 37932 USA). Other manufacturers also offers other useful radioimaging small animal scanner for example Mosaic® from Philips (Andover, Mass. 01810, USA which may be used in the practicing of this discovery.

The premise underlying the use of radioimmunotherapy in diagnosing and treating cancer is that preferential accumulation in tumor or localized tumorous region of the living mammal of a radionuclide-conjugated antibody will permit efficacious location of the tumor and selective delivery of cytotoxic radioactivity thereto and thus cause tumor regression. Hopefully this noninvasive therapy will be of immense use.

In an aspect, positron emission tomography (PET imaging) comprises detection of x-rays emitted from radionuclides that decay by positron emission and are located within the mammalian patient's body. In an aspect, PET is carried out over a time period referred to as a time course.

In an aspect, single photon emission computed tomography (SPECT imaging) comprises a collimation of gamma rays emitted by a radiopharmaceutical distribution such as detectable radioactivity emitting radiological activity within the mammalian body undergoing treatment and analysis. Generally collimators for SPECT imaging are lead and comprise thousands of various shaped parallel channels through which—and only through which—gamma rays are allowed to pass. Generally such collimators are positioned over a single crystal of NaI contained in the Gamma camera in an arrangement called an Anger camera. The image from the camera is the captured image that is presented to a human operator as part of the image in an acquisition process.

In an aspect, at least one of a PET, microPet and a SPECT image is taken of (i.e. an acquisition is made) a mammal after administration of a radiolabeled antibody to the mammal.

In an aspect, an emitting radioactive substance is produced in a process and is attached, or tagged, to an antibody as a conjugated antibody such as one that recognizes and binds to the HICSP protein and is termed labeling or radiolabeling. Once this radioactive substance is administered to a mammalian patient, emitted radioactivity localizes in the appropriate areas of the body and is detected by PET scanner.

In an aspect, images are taken over elapsed time in a dynamic fashion to assemble a developing or developed scenario of situations in a mammalian patient. This may also be referred to as a profile.

Typically an adequate and effective amount of time is allowed to elapse for the treated mammal to come to an equilibrium state following satisfactory administration of the pharmaceutical composition comprising a radioligand. Typically the mammal is placed in a position near the PET instrument or SPECT instrument allowing satisfactory operation of the PET instrument and/or SPECT instrument. The PET and SPECT instruments are capably equipped with all necessary operable computer hardware, software and operation requirements including all communication and instructive elements for full functionality. They are turned on by supplying 100 volts electric power to the instruments.

Generally after having received its administration of the radiolabeled antibody the mammal is ready for an imaging examination and is taken to an examination room that houses the PET scanner, which has an opening in the middle. In the PET scanner there are multiple rings of detectors that record the emission of energy from the radioactive substance now within in the mammal. In an aspect, the mammal is moved into the hole of the machine. In an aspect, images are obtained of the mammal as part of this radiological examination and are displayed on the monitor of a computer, suitably equipped and operably coupled to the PET scanner instrument. In an aspect, a pharmacologic assessment is made of the imaged locus of tissue.

The specific mammalian dose is calculated according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. The amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

In an aspect, a conjugated monoclonal antibody comprises immunotoxins which are made by attaching toxins (poisonous substances from plants or bacteria) to monoclonal antibodies.

Humanizing Monoclonal Antibody Therapy (Engineered human antibody) may be employed as part of the immunotherapy regime in connection with hyperthermia. In an aspect a human engineering antibody is prepared following standard laboratory procedure and is administered to a living mammal as a pharmaceutical composition.

Briefly the antibody to HICSP is prepared as described except that the part of the mouse antibody gene responsible for recognizing a specific tumor antigen (HICSP) is exchanged with other parts from a human antibody gene. The product of this mouse-human antibody gene, called a “humanized” monoclonal antibody, looks sufficiently like a normal human antibody to avoid being destroyed by the human patient's own immune system.

If desired a nonantibody based therapeutic agent may be employed as an immunotherapy in conjunction with hyperthermia with or in place of an antibody. Illustratively useful nonantibody based therapeutic agents include toxins linked to hormone-like substances referred to as growth factors.

In an aspect, immunotherapy herein comprises systemic immunotherapy which means that the immunotherapy is administered to treat the whole mammalian body. In an aspect, the immunotherapy is targeted which means that the immunotherapy is aimed at cancer cells and if possible leaves normal cells untouched. In an aspect, the antibody is targeted to tumor cells. This increases the delivery of tumoricidal doses of cytotoxic agent to tumor cells while causing a significant reduction of toxicity to normal tissues.

In an aspect, it is believed that siRNA designed according to the sequence of the gene comprising a polynucleotide having Seq. Id. No. 1 for example the siRNA having Seq. Id. No. 3 is useful to silence the gene expression in cells containing a gene comprising a polynucleotide having Seq. Id. No. 1.

In an aspect, treatment is carried out using siRNA at normal mammal body temperature or as an option may be employed in conjunction with hyperthermia. In an aspect, a gene or genes may be selectively employed to silence silenceable genes using small pieces of RNA called siRNA (short interfering RNA).

In an aspect, a suitable sequence for siRNA suitable for silencing a gene is obtained by providing Dharmacon, Inc. 1376 Miners Drive #101 Lafayette, Colo. 80026 with a suitable sequence of a gene to be silenced. Dharmacon employs a custom proprietary process to identify candidate siRHA based on the initial gene sequence from a submitter. In an aspect, siRNA having Seq. Id. No. 3 is identified by providing Dharmacon with Seq. Id. No. 1.

In a further aspect, transfectamine is obtained from InVitrogen Corporation, 1600 Faraday Avenue, P.O. Box 6482, Carlsbad, Calif. 92008 and/or Calgene Inc., 1920 Fifth Street, Davis, Calif. 95616 and a mixture is prepared in a tissue culture. This tissue culture is taken in tumor cells which “eat” the admixture comprising siRNA. Without being bound by theory it is believed that siRHA is an effective toxic agent against a gene comprising a polynuclotide having a sequence shown in Seq. Id. No. 1.

A useful web site, http://www.whitehead.mit.edu/nap/features/nap_feature_sirna.html provides technical information from Whitehead Biocomputing group which provides useful tools for identifying siRNA using a sequence of a polynucleotide such as Seq. Id. No. 1. This information is incorporated herein in its entirety by reference.

Antisense oligodeoxynucleotides (“ODNs”) or “oligos” are synthetic polymers: having e.g. monomers that are deoxynucleotides like those in DNA (“deoxyribonucleic acid”).

In an aspect, antisense oligos are synthesized for use as therapeutic agents blocking cancer disease processes by blocking the synthesis of a particular protein. In an aspect, the blocked protein comprises a polypeptide having Seq. Id. No. 2. Such blocking would be achieved by the binding of the ODN to the mRNA from which that protein (polypeptide having Seq. Id. No. 2) is normally synthesized.

In aspect ODN's are in a suitable vector for competent transfection into the tumor cells having a gene comprising a polynucleotide having Seq. Id. No. 1.

The construction of a suitable vector can be achieved by any of the methods well-known in the art for the insertion of exogenous DNA into a vector. see Sambrook et al., 1989, Molecular Cloning, A Laboratory Manual, Cold Spring Harbor Press, N.Y.; Rosenberg et al., Science 242:1575-1578 (1988); Wolff et al., PNAS 86:9011-9014 (1989). For Systemic administration with cationic liposomes, and administration in situ with viral vectors, see Caplen et al., Nature Med., 1:39-46 (1995); Zhu et al., Science, 261:209-211 (1993); Berkner et al., Biotechniques, 6:616-629 (1988); Trapnell et al., Advanced Drug Delivery Rev., 12:185-199 (1993); Hodgson et al., BioTechnology 13:222 (1995).

In an aspect, a non-invasive method of using products of a gene comprising a polynucleotide having a sequence shown in Seq. Id. No. 1 as an indicator of the effectiveness of administered therapy to a mammal in the treatment of cancer comprises: applying heat to a tissue locus containing a suspected tumor in an amount and for amount of time effective to activate the gene and forming a treated tissue locus; administering a therapy to the mammal; obtaining a sample of the locus and analyzing the sample for the extent of presence of a protein comprising a polypeptide having a sequence shown in Seq. Id. No. 2 and determining that the extent provides an indication of the therapy effectiveness of the administered therapy. In an aspect, the extent is used to determine the prophylactic effect of an administered therapy. In an aspect the administered therapy is a drug.

In an aspect the drug is a candidate drug for testing. In an aspect a candidate drug is determined to be toxic to benign and malignant tumors and precancerous lesions when there has been a detectable decrease in the number of benign and malignant tumor and precancerous lesion cells following treatment with the candidate drug as compared to the number of benign and malignant tumor and precancerous lesion cells prior to treatment.

While the term “sample” envisions performing the analysis of this discovery on an entire mammal, it is contemplated that α-diagnosis may be performed on a representative sample thereof. For example a tissue biopsy sample can be utilized in the practice of this invention. However, the conditions of such utilization would be such that the sample would have remained and remain viably life sustaining for the living mammal.

In an aspect, a method of reducing at least one of the size and number of tumor cells in a living mammal comprises administering to the mammal an effective amount of an antibody which binds to a protein comprising a polypeptide having sequence shown in Seq. Id. No. 2. In an aspect, the antibody is coupled to a tumor cytotoxic agent.

In an aspect, a method of evaluating the tumor bearing potential of a mammal comprises hyperthermically increasing the expression of a protein comprising a polypeptide having a sequence shown in Seq. Id. No. 2 in the mammal and analyzing for increased expression of the protein indicative of a higher tumor bearing potential.

In an aspect, proteins induced and/or accumulated on cell surface by heating and used as target in hyperthermia-based tumor-targeting therapy.

The following examples are illustrative but are not meant to be limiting of the invention in any way.

### EXAMPLE 1

Isolation of a novel gene and its encoded heat-inducible cell surface protein (HICSP).

### Example 1A

Background of discovery of the novel gene and its encoded protein HICSP (heat-inducible cell surface protein)

NSY42129 (NSY) cell line was established from a human colon cancer and initially characterized (Mai Xu, Establishment and initial characterization of NSY42129 human colon adenocarcinoma cell line. 1992, Journal of China Medical University, Vol.21 (1): 125-136).

The inventor cultured NSY cells in an incubator at a temperature of 41° C. and surprisingly found that NSY cells could continuously grow at the elevated temperature. Eventually NSY cells have been maintained at 41° C., passed for many passages and became a permanent variant (sub-cell line) of NSY cell line. To distinguish the variant from its parental NSY or wild type cells the variant cells continuously maintained at 41° C. was designated as NSY-CHR (NSY-chronic heat resistant).

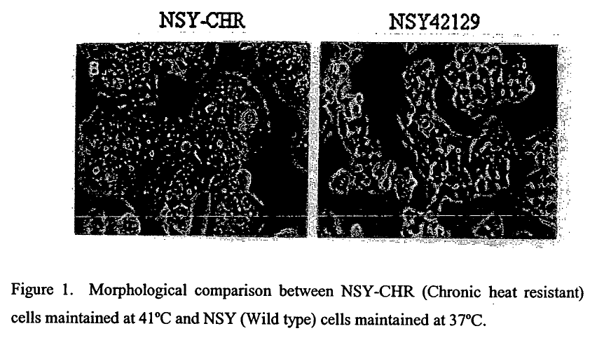

Comparing the morphology between NSY cells cultured at 37° C. and NSY-CHR cells maintained at 41° C., NSY cells grow as a single cell mono-layer typical of most adherence dependent cell lines as shown in right panel of FIG. 1, while NSY-CHR, left panel of FIG. 1, grow as multi-cellular colonies which display an outer envelop like structure indicated by black arrows.

Based on the morphological features of NSY-CHR cells we hypothesized that the outer envelope structure of NSY-CHR cells may play an important role in heat resistance.

To study the mechanisms of thermal (heat) resistance, the inventor explored the unusual structures (envelope like structure) of NSY-CHR cells at molecular level.

As a cell surface molecule, E-cadherin, a trans-membrane protein and main component of cell membrane in epithelial cells has been extensively studied. Therefore, the inventor decided to measured E-cadherin expression in NSY and NSY-CHR cells by RT-PCR assay.

### Example 1B

Inventor's Discovery of a novel gene DNA fragment

RT-PCR assay:

Material and experimental procedures:

To measure E-cadherin expression in NSY-CHR cells at transcriptional level, total RNA was isolated following standard procedures using TRI reagent and BCP (1-bromo-3-chloropropane) purchased from Molecular Research Center INC (Cincinnati Ohio). RT-PCR assay was performed using Access RT-PCR Introductory System (Kit), Cat. No A1260, from Promega Corporation (Madison, Wis.) with primers synthesized by Nucleic Acid Chemistry Laboratory according to the sequence of 5° CCTTCCTCCCAATACATCTCCC3′ and 5′TCTCCGCCTCCTTCTT CATC3′. This pair primer was designed for E-cadherin. The PCR products were resolved on 0.7% agar gel.

Results:

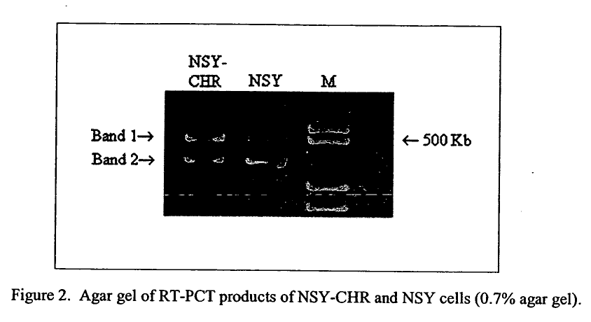

FIG. 2. 0.7% agar gel of RT-PCR products of NSY and NSY-CHR cells. In FIG. 2, first line from the left is NSY-CHR mRNA RT-PCR product, the second (middle) line is NSY mRNA RT-PCR product and the last line, M, on the right is a marker of nucleotide size.

As shown in FIG. 2, two bands, Band 1 with 500 Kb and Band 2 with less than 500 Kb, were observed in both NSY-CHR and NSY cells, but Band 1 significantly increased in NSY-CHR cells.

One of these bands is E-cadherin and the other band is a novel one. Based on these results we are not sure which one is E-cadherin and which one is a novel one.

Initially the inventor tried to measure E-cadherin expression in heat resistant NSY-CHR cells. However instead the inventor isolated, characterized and identification of the novel gene and its novel product (protein). To further confirm the existence of novel gene t northern blot was performed using the PCR products as a probe.

Northern blot:

Material and experimental procedures:

RNA isolation: as described before total RNA was isolated following standard procedures by using TRI reagent and BCP (1-bromo-3-chloropropane) purchased from Molecular Research Center INC (Cincinnati Ohio). About the northern blot, briefly the procedures are as follows: a) Prepare 1% agar gel with 10 ml 10× Mops buffer and 3 ml formaldehyde. b) Sample preparation, 10 μg RNA from NSY and NSY-CHR cells plus 11 μl H₂O and 39 μl Loading mix. Then the samples were autoclaved for 15 minutes at 55° C. c) Resolve RNAs on 1% agar gel with 90 V for 2-3 hr. d) Transfer the RNA onto Genescreen plug membrane (Nylon, DuPont) for overnight. d) Hybridize overnight with ³²P labeled cDNA (PCR product) probe at 42° C. e) Finally scan with Storm-image 840 (Molecular Dynamics INC, USA).

Results:

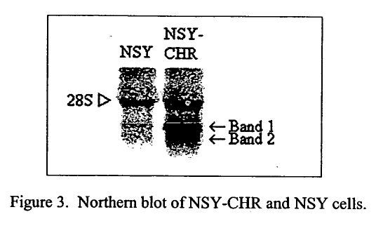

FIG. 3. Northern blot of NSY and NSY-CHR cells:

Left line is NSY cell and the right line is NSY-CHR cell. It clearly showed two intensive bands in NSY-CHR cells just below the 28s RNA band on the blot. Both bands are significantly increased in NSY-CHR cells as compared to the bands from NSY cells. So these results indicate that E-cadherin was increased at the transcription level and mRNA of the novel gene fragment not only exists, but also increased in NSY-CHR cells.

Sequencing the DNA fragment of the novel gene

To analyze the new fragment of gene, the cDNA (RT-PCR products) of novel gene fragment was cloned into pPCR-Scrip AMP SK(+) cloning vector and transfected into XL10-Gold Kan Ultracompetent cells. After purifying the RT-PCR products with StrataPrep PCR purification kit, polishing the purified PCR products and inserting the cDNA into pPCR-Scrip AMP SK(+) cloning vector, finally the vector was transfected into competent cells.

In detail cloning procedures are as follows. 1) Prepare ligation reaction solution, a) 1λ of pPCR-Scrip SK(+) cloning vector (10 ng/μl); b) 1λ of PCR-Scrip 10× buffer; c) 0.5λ of 10 mMrATP; d) 4λ blunt-ended PCR product; e) 1λ SrfI restriction enzyme (5 μ/μl); f) 1λ T4 DNA ligase (4 μ/μl); 1.5λ distilled water. 2) Mix the ligation reaction solution gently and incubate this reaction for 1 hr at room temperature. 3) Heat the ligation reaction solution for 10 minutes at 65° C. 4) Store the ligation reaction on ice until ready to use for transformation into the Epicurian Coli XL10-gold Kan Ultra-competent cells.

Transfection of the cloned vector into E. Coli XL 10-gold Ultra-competent cells (QiaGen):

a) Thaw the XL 10-gold Ultra-competent cells on ice; b) Gently mix the cells by hand and aliquot 40λ of the cells into a chilled 12 ml falcon 2059 polypropylene tube; c) add 1.6 λ of XL10-gold β-mercaptoethanol mix to the 40λ Ultra-competent cells; d) Swirl the contents of the tube gently and incubate on ice for 10 minutes, swirling gently every 2 minutes; e) incubate the tube on ice for 30 minutes; f) Heat pulse the tube in 42° C. water bath for 30 seconds; g) incubate the tube on ice for 2 minutes. h) Add 0.45 ml preheated NZY-broth to the tube and incubate for 1 hr at 37° C. with shaking. i) Grow the transfected cells in plate and incubate for overnight at 37° C. for blue-white color screening. Colonies containing plasmids with insert will remain white. j) Expanding the cell culture for DNA preparation and sequencing.

ABI PRISM BigDye Terminator Cycle Sequencing:

Preparation of DNA for sequencing, For DNA sequencing the first step is to isolate or purify DNA from the plasmid, which contains the new gene fragment by using plasmid Mini purification Kit (Cat. No. 12123 QIAGEN Miami, Fla., USA).

The procedures are as follows. 1) Resuspend the bacteria pellet in 0.3 ml of buffer P1 (from the mini purification kit). 2) Add 0.3 ml of buffer P2, mix gently and incubate at room temperature for 5 minutes. 3) Add 0.3 ml of chilled buffer P3, mix immediately but gently, and incubate on ice for 5 minutes. 4) Equilibrate a QIAGEN-tip 20 by applying 1 ml Buffer QBT (from the kit) and allow the column to empty by gravity flow. 5) Apply the supernatant from 4) to the QIAGEN-tip 20 and allow it to enter the resin by gravity flow. 6) Wash the QIAGEN-tip 20 with 4×1 ml buffer QC. 7) Elute DNA with 0.8 ml buffer QF. 8) Precipitate DNA with 0.7 volumes of room temperature isopropanol. Centrifuge immediately at 10,000 rpm for 30 minutes in a micro-centrifuge and carefully decant the supernatant. 9) Wash DNA with 1 ml of 70% ethanol, air dry for 5 minutes, and re-dissolve in a suitable volume buffer. DNA sequence, ABI PRISM BigDye Terminator Cycle Sequencing Ready Reaction Kit was used in this test. (ABI PRISM BigDye Primer Cycle Sequencing Kits provide optimized fluorescent primers. for decreased background noise in an wide array of sequencing applications. )Applied Biosystems, 850 Lincoln Centre Drive, Foster City, Calif. 94404 USA

As two main PCR products were cloned and transfected into competent cells, as shown in FIGS. 2 and 3. DNA samples from multiple clones were used for sequencing. The chance for each PCR product is 50%. One is E-cadherin and the other one is a novel species. In this test we pick up two bacteria clones transfected with the RT-PCR products, 2E-5 and 2E-7. Briefly the procedures are as the following.

Terminator ready reaction mix-8λ.

DNA template from 2E-5 clone-2λ.

Primer #1 upsteam T3-5λ.

De-ionized water—4λ.

40 mM MgCl—1λ.

Terminator ready reaction mix-8λ.

DNA template from 2E-5 clone-2λ.

Primer #2 upsteam T7-5λ.

De-ionized water-4λ.

40 mM MgCl-1λ.

Terminator ready reaction mix-8λ.

DNA template from 2E-7 clone-2λ.

Primer #1 upsteam T3-5λ.

De-ionized water-4λ.

40 mM MgCl-1λ.

Terminator ready reaction mix-8λ.

DNA template from 2E-7 clone-2λ.

Primer #2 upsteam T7-5λ.

De-ionized water-4λ.

40 mM MgCl-1λ.Run 25 cycles of the following: 1) 96° C. for 10 seconds, 2) 50° C. for 5 seconds and 3) 60° C. for 4 minutes. Then rapid thermal ramp to 4° C., hold until ready to purify and then send the PCR products to PNACL-DNA Sequencing Center at Medical School of Washington University. PNACL, Biotechnology Center, Fourth Floor, 4559 Scott Ave, St. Louis, Mo. 63110, This protein and peptide sequencing service utilizes the advanced instrumentation to provide routine sequencing at the very low (1-5) pmol level. Sequencing can be performed on either soluble samples or from samples blotted on to PVDF.

Results

The DNA sequencing results showed that the sequence from 2E-7 clone is E-cadherin. The other sequence from 2E-5 clone is a DNA fragment of the novel gene. A total of 408 bases of the inventor discovered novel gene DNA fragment and its encoded peptide with 136 amino acid, were blasted in NCBI databases (National Center for Biotechnology Information National Library of Medicine Building 38A Bethesda, Md. 20894 including all GenBank, EMBL, DDBJ, PDB, and Swiss.

The gene and protein banks search showed that both sequence of the identified DNA fragment and the DNA fragment encoded peptide are more than 95% matching BAT2 (HLA-B associated transcription factor 2), but not 100% .

To distinguish using nomenclature the inventors novel gene encoded protein from BAT2 the novel gene encoded protein was designated as heat-inducible cell surface protein (herein referred to throughout as “HICSP”).

### EXAMPLE 2

Expression of the novel gene encoded protein HICSP (heat-inducible cell surface protein) was increased on the surface of NSY-CHR (NSY-chronic heat resistant) cells

Material and Methods

Antibody:

Generation of rabbit Polyclonal antibodies: To further characterize his novel gene product the inventor generated rabbit polyclonal antibodies against a short peptide synthesized according to the sequence of 136 amino acids from the novel gene encoded protein. The Rabbit polyclonal antibody against the short peptide was designated as 805 k.

In details, 805 k rabbit polyclonal antibody was generated with immunization of a short peptide of HICSP. The procedures to generate rabbit polyclonal antiserum are as follows: 1) synthesized peptides according to the sequence of HICSP protein. 2) Immunized the rabbit with the synthesized peptide. 3) Test the serum with Elisa method to determine if antibodies specific to the peptide were induced in immunized rabbit. 4) If the titer of antibody reacting to the antigen (the peptide) is high enough (1:4,000×) then the antiserum was taken from the immunized rabbit.

For measuring cell surface protein two methods were applied. One method was immunofluorescence staining on living cell system. The other method was biotin-streptavadin precipitation technique.

Immunofluorescence staining in living cells of tissue culture (serological assay): This staining takes advantages of serological assay that only permits the detection of cell surface proteins and not cytoplasm proteins in the living cell system due to the fact that antibodies cannot penetrate the membrane of living cells into the cell cytoplasm. The assay procedures described as follows, cells growing in 24 well plat were washed three times with PBS and briefly dried with paper towel. The cells were incubated with different concentration of rabbit polyclonal antibody against HICSP for 40 minutes at 37° C. incubator. Then the cells were washed three times, briefly dried again, incubated with FITC conjugated goat anti-rabbit antibody at a dilution of 1:500 (Jackson Immuno Research Laboratories, P.O. Box 9872 West Baltimore Pike West Grove, Pa., USA 19390) and kept at 37° C. for 40 minutes. After washing three times the cells are ready to be observed under fluorescence microscope.

Biotinylation assay, briefly cell surface proteins were labeled by biotin and precipitated by streptavidin conjugated to agarose. Components of the precipitates are cell surface proteins. Detail procedures are described as follows;

Cell lines: human colon adenocarcinoma NSY or its variant NSY-CHR cells were cultured in T25 cm² with RPMI 1640 medium supplemented with 10% fetal calf serum, 50 units/ml sodium penicillin G and 50 μg/ml streptomycin sulfate.

Buffers and reagents:

PBS/CM, Phosphate buffered saline containing 1.0 mM MgCl₂ and 1.3 mM CaCl₂. Solfo-NHS-biotin solution, 0.5-mg/ml in PBS/CM buffer, EZ-Link™ sulfo-NHS-LC-Biotin (Pierce Biotechnology Inc, Customer Service Department, P.O. Box 117 Rockford, Ill. 61105 U.S.A,

50 mM NH₄Cl in PBS/CM: for 100 ml, 0.26 g NH₄Cl, 1 ml 1.3 M CaCl₂, 1 ml 1.0M MgCl₂ to 48 ml H₂O, The final volume should be 100 ml.

TPI Lyses buffer: 1) 1 ml Triton X-100, 2) 0.242 g Tris base, 3) 1 ml 0.5M EDTA, 4) 0.847 g NaCl, 5) 0.2 g Bovine albumin. Add 90 ml H2O, adjust PH to 8.0 with NaOH or HCl, and then adjust the volume to 100 ml, store at 4° C. Add 100 μg PMSF and 10 μg proteinase inhibitor cocktail to 10 ml lyses buffer.

TPII: 0.1% SDS, 20 mM Tris, 150 mM EDTA, PH 8.0 containing 0.2% BSA: For 500 ml, 5 ml 10% SDS, 1.21 g Tris base, 5 ml 0.5M EDTA, 4.235 g NaCl, 1 g BSA, and 450 ml H₂O, adjust PH to 8.0 with HCl, then adjust the volume to 500 ml with dd H₂O. Store at 4° C.

TPIII: 20 mM Tris, 150 mM NaCl, 5 mM EDTA PH 8.0 containing 0.2% BSA. 1.21 g tris base, 5 ml 0.5M EDTA, 4.235 g NaCl, 1 g BSA and 450 ml H₂O. Adjust PH to 8.0 with HCl, and then adjust the volume to 500 ml with dd H₂O, store at 4° C.

TPIV: 50 mM Tris, PH 8.0, For 500 ml, 3.0725 g Tris base and 450 ml dd H₂O, Adjust PH to 8.0 with HCl, then adjust the volume to 500 ml with dd H₂O, store at 4° C.

Immobilized Streptavidin (Pierce Biotechnology Inc., Rockford, Ill.)

Test Procedures

Biotinylation of cell surface protein, 1) prepare exponential growing cells in T25 cm2 flask. 2) Wash cells in the flask with ice cold PBS/CM for 3 times. 3) Add 2 ml fresh solution of sulfo-NHS-biotine and incubate with gentile shaking at room temperature for 20 minutes. 4) Repeat step 3. 5) Quench the reaction with removing the solution and add 2 ml of 50 mM NH4Cl in PBS/CM and incubate with gentle shaking for 10 minutes at 4° C. 6) Rinse twice with PBS/CM. 7) Add 1 ml of lyses buffer. 8) Centrifuge lysate in a micro-centrifuge at 4° C. at 13,000 g for 10 minutes, collect the supernatant and discard the pellet. 9) Add 250-500 μl immobilized streptavidin to each sample. 10) Incubate 40 minutes at room temperature. 11) Centrifuge the agarose complex in 13,000 for 1 minute, remove the supernatant and resuspend the beads in 1 ml TPI buffer. 12) Wash three times with TPII. 13) Wash three times with TPfi. 14) Wash one time with TPIV. 15) Add 200 μl Leammli sample buffer and autoclave for 5 minutes. 16) Run One D SDS-PAGE and transfer the proteins onto PVDF membrane. 17) The blot was probed with 805 k rabbit polyclonal antibody.

Results:

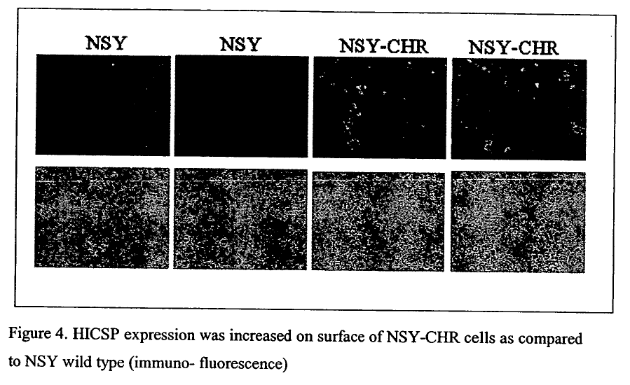

FIG. 4. Immunofluorescence staining of NSY and NSY-CHR cells using 805 k rabbit polyclonal antibody with dilution of 1000×. It showed that expression of HICSP increased in NSY-CHR cells as compared with its wild type NSY cells.

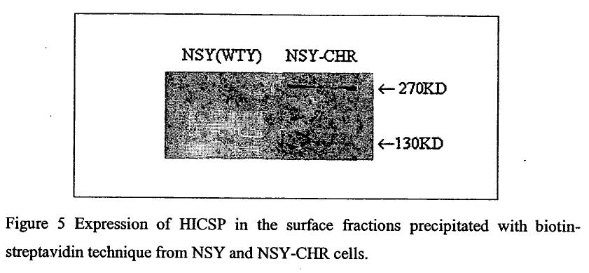

FIG. 5. Western blot of cell surface proteins precipitated by biotin-streptavidin from NSY and NSY-CHR cells: The biotin-streptavidin precipitated samples that only contain cell surface proteins were electrophoresesed through 7% SDS PAGE gel and transferred onto PVDF membrane.

SDS PAGE means SDS POLYACRYLAMIDE GEL ELECTROPHORESIS, see htto://www.mcb.uct.ac.za/sdspage.html

(PVDF means KYNAR® Polyvinylidene Fluoride (PVDF) and KYNAR Flex® PVDF are highly chemically resistant fluoropolymers and are registered trademarks of the duPONT Company, Wilmington, Del., USA.

The membrane was probed with 805 k rabbit polyclonal antibody. It showed that 805 k polyclonal antibody binds to 2 protein bands with molecular weight of 130 KD and 270 KD. Both proteins with molecular weight of 270 KD and 130 KD are increased in NSY-CHR cells. The protein with 270 KD is more prominent. Therefore 130 KD protein may be premature type or degrader of 270 KD protein.

### EXAMPLE 3

The novel gene encoded protein HICPS is not BAT 2 protein but that gene sequence is similar, but not the same to that of the isolated novel gene.

To clearly distinguish the novel gene product carrying a polynucleotide having Seq. Id. No. 1 from BAT2 protein, whole cell proteins (lysates) were analyzed by western blot with 805 k rabbit polyclonal antibody against peptide synthesized according to the amino acid sequence of HICSP.

Method of western blot (for detail procedures please see Example 4):

Briefly the whole cell lysates including components of nucleus, cytoplasm and cell membrane from NSY, NSY-CHS and NSY-CHR cells were successfully resolved on 7% SDS-PAGE gel and transferred onto PVDF membrane. The blot was incubated with 805 k rabbit polyclonal antibody. NSY-CHS (NSY Chronic heat sensitive) is a variant of NSY cells.

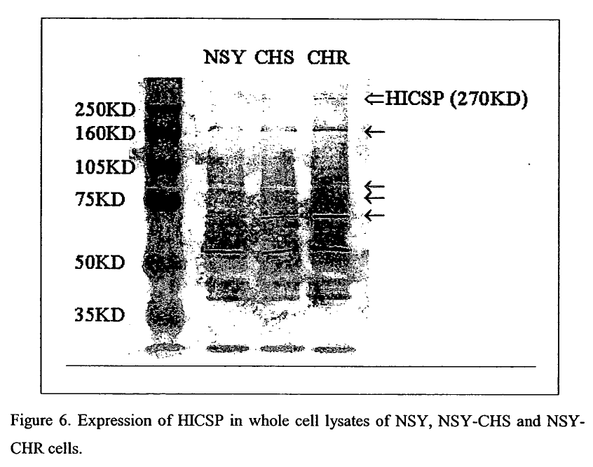

FIG. 6. Expression of HICSP in NSY, NSY-CHS and NSY-CHR cells: There are several protein bands altered (increase or decrease) in comparing NSY and NSY-CHR cells. The protein with molecular weight 270 kD were detected and increased in NSY-CHR cells, but the 130 kD protein was not observed probably due to the limited amount of this protein in whole cell lysate. Protein bands, especially 170 kD protein detected by rabbit polyclonal antibody 805 k in whole cell lysates are cytoplasm forms of HICSP or cross-reaction. (Note k=1,000)

In summary, the whole gene has not been completely cloned but the protein analysis is compelling evidence that HICSP is novel and unique. This additional evidence supports the novelty and patentability of the discovery herein presented: 1) the molecular weight of HICSP (270 kD and/or 130 kD) is different from that of BAT 2 protein which contains about 2157 amino acids with a molecular weight of 227 kD (NiceProt view of Swiss-Prot: P48634 and Banerli at el, proc. Natl. Acad. Sci. USA 87:2378, 1990). HICSP is 270 kD and/or 130 kD. 2) HICSP is expressed on the cell surface, while large prolin-rich protein BAT2 showed that neither a hydrophobic leader nor an obvious transmenbrane region are appear (Banerji at el, proc. Natl. Acad. Sci. USA 87:2378, 1990). 3) BAT2 protein is limited to leukemia cell lines tested so far, but HICSP expressed on the surface of tumor cells derived from epithelial cells. 4) BAT2 gene contains about 6914 bases and 2157 amino acid. Only part of the pat of the novel gene, 408 bases, and HICSP protein, 136 amino acids, is similar to BAT2 gene and its encoded protein, but not exactly matching. 5) HICSP is stress including heat-inducible and/or accumulative on cells surface. Therefore the isolated, purified and characterized HICSP gene and its encoded protein are novel and unique.

### EXAMPLE 4

The isolated novel gene and its product HICSP are ideal target for stress, including hyperthermia, based tumor-targeting therapy.

### EXAMPLE 4A

HICSP over expression and/or accumulation after heating at 41° C. for a limited time: (Analyzed by biotin-labeling cell surface proteins and streptavidin precipitation technique and western blot).Gottadi, C and Caplan M (1993). Cell surface biotinylation in the determination of epithelial membrane polarity. J. Tissue Cult. Meth. 14, 173-180. Hanzel. D., Nabi. I. R., Zurolo, C., Powell, S. K., and Rodriguez-Boulan, E., (1991) New techniques lead to advances in epitherlial cell polarity. Semin. Cell Biol. 2, 341-353. Laemmili, U.K., (1970). Cleavage of structural proteins during the assembly of the head of bacteriophage T4. Nature 227,680-685.

Methods: please see EXAMPLE 2 ABOVE

Results for Example 4

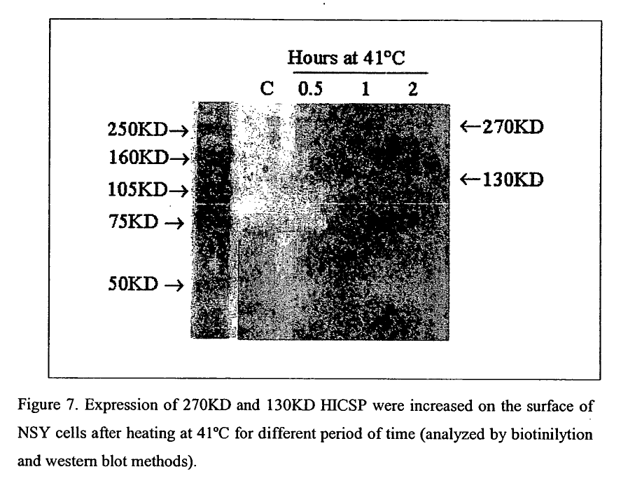

FIG. 7 showed HICSP with molecular weight of 270 KD and 130 KD were increased and/or accumulated in cell membrane fractions after heating at 41° C. for an indicated time.

### EXAMPLE 4B

Expression of HICSP in tumor and normal cells after heating at 41° C. (Analyzed by western blotting). Due to 130 kD HICSP was hardly detectable in whole cell lysates, therefore the following HICSP analysis in tumor and normal cell lines is 270 kD HICSP. Material and methods: Tumor cell lines: NSY cell line was derived from a human colon adenocarcinoma (Mai Xu et al. 1996, Int. J. Hyperthermia 12(5): 645-660). Human colon adenocarcinoma HT29 and HCT15, human breast carcinoma MCF7, human prostate carcinoma PC3, and human glioblastoma. All cell lines, except NSY and HCT15 cells that were maintained in RPMI1640 medium, were cultured in DMEM medium and obtained from ATCC (American Type Culture Collection, P.O. Box 1549, Manassas, Va. 20108,USA or Rockville, Md., USA), unless otherwise noted. The tissue culture mediums were supplemented with 10% fetal calf serum, 50-units/ml sodium penicillin C and 50 mg/ml streptomycin sulfate. Cells were cultured at 5% CO₂ humidified and water jacketed incubator (Forma Scientific INC, P.O. Box 649, Marietta, Ohio 45750.

None tumor (normal) cell lines:

CRL7483, BNL-C2 and MRC-5 are known human normal fibroblast cell lines. All cell lines were obtained from ATCC (American Type Culture Collection, P.O. Box 1549, Manassas, Va. 20108, USA or Rockville, Md.). CRL7483 was cultured in DMEM, BNL-C2 and MRC5 in MEM medium. All the cultured mediums were supplemented with 10% fetal calf serum, 50-units/ml sodium penicillin G and 50-mg/ml streptomycin sulfate.

Heat treatment and western blotting cells cultured in T25 flasks at 5% humidified 37° C. incubator. For heat treatment the cultures were shifted to 41° C. incubator from 37° C. for indicated time. The cells in T25 flask was washed three times with cold PBS and collected by scraping with scraper. The cell pallet was sonicated with 2 Watts of output for 30 to 40 seconds with 60 Sonic Dismembrator (Fisher Scientific). Protein content of the cell lysate was quantified using BIORAD protein assay. Mixing equal volume of cell lysate with 2× 1 D sample buffer and autoclaving at 95° C. on heating plate for 5 minutes. Equal amount of protein was loaded and resolved on 7% SDS PAGE gel. The proteins on SDS PAGE gel were transferred onto PVDF membrane. The membrane was incubated with blocking buffer containing 5% dry milk and then with primary antibody 805 k rabbit polyclonal anti serum against HICSP with the dilution of 1:500 at room temperature for 1 hr. After washing three times with TBST buffer, the membrane was incubated with secondary antibody alkaline phosphatase conjugated donkey anti rabbit antibody with 1:1000 dilutions for 40 minutes at room temperature. After washing the membrane for four times with PBST the membranes were developed with a substrate, BCIP/NBT solution (Sigma-Aldrich Corp. St. Louis, Mo., USA.), for 3 minutes to 6 minutes. Finally the membrane was washed with tap water to stop the reaction of the substrate with alkaline phosphatase.

Results:

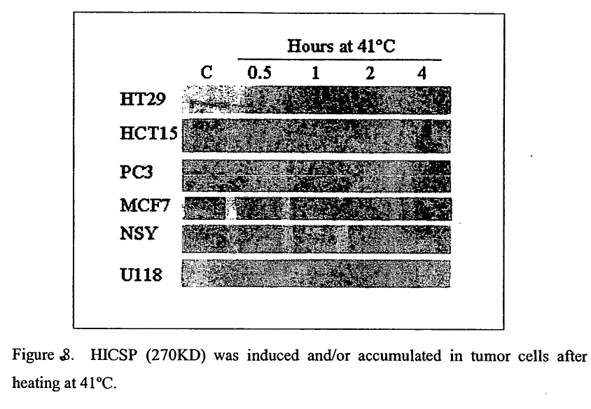

FIG. 8 depicts that HICSP (270 KD) was induced and/or accumulated in all tumor cell lines tested after heating at 41° C. HICSP (270 KD) induced by heating at 41° C. for as little as 0.5 hr and reached maximal amount at 1 or 2 hr heating at 41° C.

Table 1. Shows data from the Inventors quantitative analysis of HICSP expression after heating at 41° C. in tumor cells tested.

Table 1 below showed HICSP protein (270 kD) induction and/or accumulation after heating at 41° C. for different period of time in human malignant tumor cell lines derived from different origins. The HICSP 270 kD protein bands on the blot membranes were quantified by densitometor equipped with Imagequant software (Molecular Dynamics Inc). The OD value from heated samples was normalized by that from controls. The number showed in heated samples is fold increase in HICSP protein after heating.

TABLE 1
quantitative analysis of 270 kD HICSP in tumor cells.

| | | | Hours at 41° C. | | | |
|---|---|---|---|---|---|---|
| Cell line | Origin | Control | 0.5 hr | 1 hr | 2 hr | 4 hr |
| HT-29 | Human colon ca. | 1 | 1.1 | 1.6 | 1.36 | 0.48 |
| HCT15 | Human colon ca. | 1 | 9.5 | 20 | 21 | 11.6 |
| PC3 | Humanprostate ca. | 1 | 2.7 | 1.9 | 1.4 | 1 |
| MCF7 | Human breast cancer | 1 | 1.3 | 1.4 | 2.1 | 1.9 |
| NSY | Human colon ca. | 1 | 1.2 | 1.1 | 0.6 | 0.96 |
| U118 | Human glioblastoma | 1 | 2.2 | 2.5 | 2.8 | 3.2 |

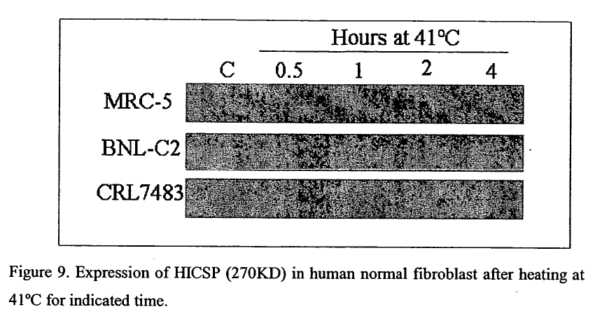

FIG. 9 depicts that HICSP was immeasurable in human normal fibroblast by western blot before and after heating.

### Example 4C

HICSP was localized on tumor cell surface and increased after heating at 41° C.—Analyzed by immunofluorescence staining method.

Material and methods:

Tissue culture preparation and Hyperthermia treatment:

80% confluent cells monolayers were trypsinized and the cells were re-cultured in 72 Microwell plate. 800 cells are seeded in each micro well. The cultures were maintained at 37° C. for about 24 up to about 48 hr and then shifted to the 41° C. incubator in which chamber temperature was calibrated by conventional thermometry using a National Bureau of Standards-traceable Thermometer (ERTCO, New York, N.Y., USA) and by using a Luxtron 3100 fluoroptic thermometer (Luxtron Corp., Santa Clara, Calif., USA).

Results:

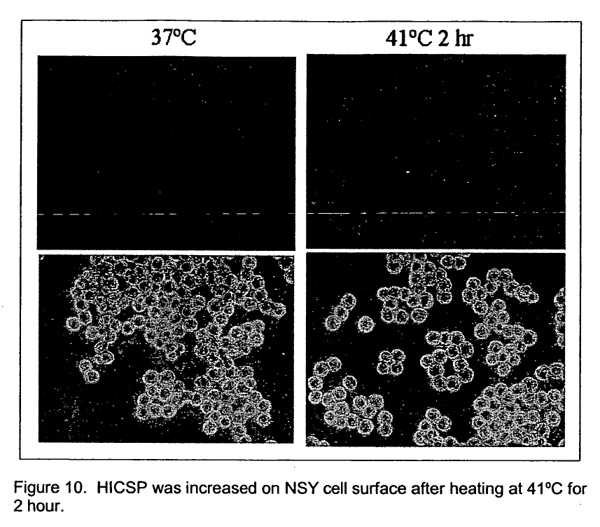

FIG. 10 depicts HICSP localized on the surface of human colon adenocarcinoma NSY tumor cells. It clearly demonstrated that HICSP was localized on cell surface and increased its expression after heating at 41° C. for an indicated time. Please also see table 1.

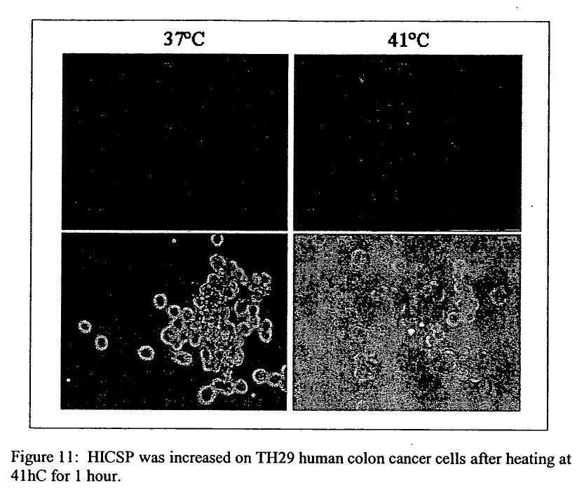

FIG. 11 depicts HICSP localized on the surface of human colon adenocarcinoma HT29 tumor cells. It also demonstrated that HICSP was observed on cell surface and increased its expression after heating at 41° C. for an indicated time. Please also see table 1.

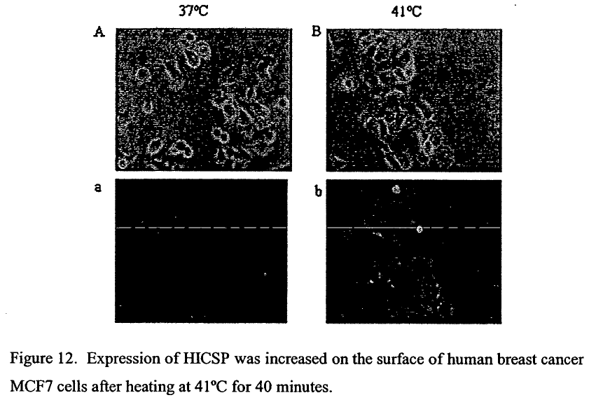

FIG. 12 depicts HICSP expression on the surface of human breast cancer MCF7 cells after heating at 41° C. for 40 minutes. The cells were stained with 805 k rabbit anti serum against HICSP with 100 dilutions. Panel A and B are bright light to show where the cells are. Panels a and b are depicts the results of using UV light to demonstrate HICSP. In this figure, panel A is the same field as panel a and panel B is the same field as panel b. The dark field of panel a indicate that no HICSP was detected. In panel b bright green staining with a ring shape indicates that HICSP increased and/or accumulated on the cell surface after heating at 41° C. for 40 minutes. The images in panels a and b were taken by using the same settings, such as brightness and exposure time.

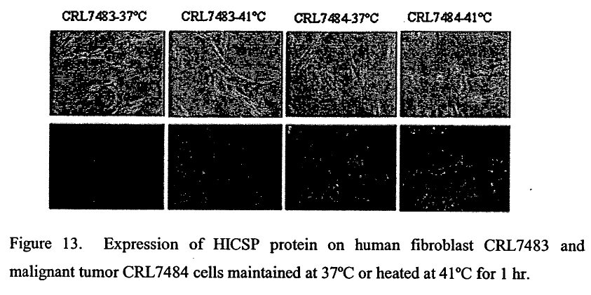

FIG. 13 depicts that HICSP expression on human normal fibroblast and malignant tumor cells maintained at 37° C. or heated at 41° C. for 1 hr. CRL7483, normal fibroblast and CRL7484 breast cancer cells originated from the same patient as a pair of normal and malignant cell lines purchased from ATCC. In this figure the upper panel is a brightfield exposure and the lower panel is UV light excitation. The images from UV light exposure were taken under the same exposure conditions. The images show that more HICSP (bright with WV light exposure) was detected on the tumor cell surface than on normal fibroblasts. After heat treatment at 41° C. for 1 hr the amount of HICSP increased and/or accumulated at the tumor cell surface, but not on normal fibroblasts (the field in the CRL7483-41° C. panel is dark under UV excitation).

### EXAMPLE 5

Expression of HICSP was successfully induced and/or accumulated by a lower heating temperature of 40° C. in HT29 living human colon adenocarcinoma cells.

Material and method

Western blot, please see EXAMPLE 4A (Above)

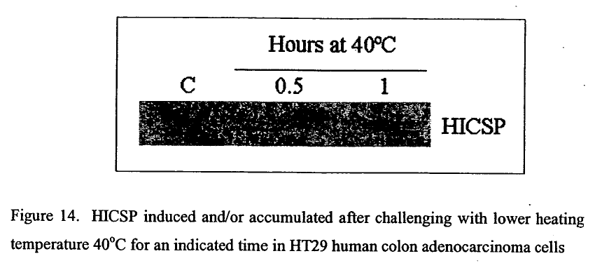

FIG. 14 depicts that HICSP were over expressed and/or accumulated after heating at 40° C. for an indicated time in HT29 cells. The amount of HICSP significantly induced and/or accumulated in HT29 cells after heating for 1 hr at 40° C.

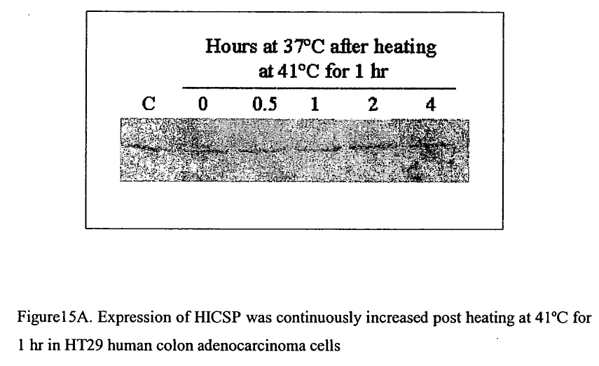

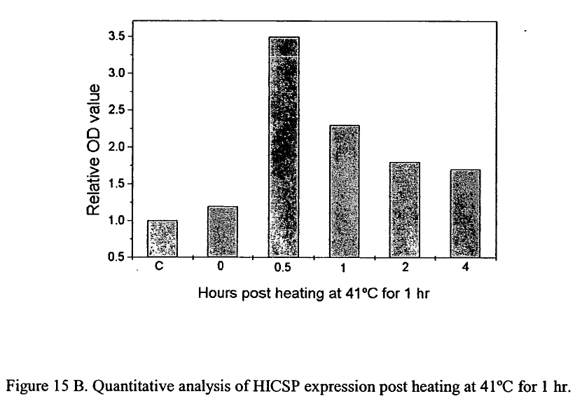

FIG. 15A depicts that HICSP was increased and/or accumulated up to 3 to 4 hours of after heating.

FIG. 15 B depicts quantitative analysis of HICSP post heating at 41° C. for 1 hr. It showed that HICSP continuously increased and/or accumulated after heating and reached about 3.5 fold after 30 minutes heating. After 1 and 2 hr heating the amount of HICSP was still about 2-fold higher than that from control.

### EXAMPLE 6

Method of hyperthermia-based tumor successfully immunotargeting therapy to treat patients with benign and malignant neoplasms and pre-cancerous lesions

Local, regional and whole body hyperthermia-based tumor immunotargeting therapy with temperature of 41° C. for 1 hr heating will be successful because HICSP was induced and/or accumulated in tumor cells tested and with the heating time as little as 0.5 or 1 hr. If patients cannot tolerant a higher temperature, then 40° C. hyperthermia is also effective to induce and/or accumulate the expression of HICSP.

In an aspect administration of the conjugated antibody is done at a time in the range from about 30 minutes to about two hours past the time when a patient being treated has had his/her core body temperature elevated to about 41 degrees C. for about one hour. In practicing the invention those of skill in the art will carry out the administration commensurate with the teachings of this specification and of the patient situation and the diagnosis and treatment being carried out and will fake into account any patient concerns. Normally the timing of the administration is such that the passage of ample time is desired sufficient to have conditions present such that expression of the protein would have been conducive. Generally the amount of conjugated antibody is administered is an effective amount such as at 30 to 60 mCi/m2. This dose is based on the radiation dose not protein. About 1 mg to about 5 mg of antibody should be fine for each treatment. This is usually repeated as the same treatment for about 5 to about 8 times until HAM (human against mouse antibody) happens.

Advantages of this novel hyperthermia-based immunotargeting therapy by using the isolated heat-inducible cell surface protein (HICSP) as target for cancer treatment are: 1) increase specificity of biological markers to tumor cells, e.g. the antigen expressed or increased is limited to cells in heated area or heated cells (The local heating device is presently available in the clinic); 2) enhance antigen expression homogeneously in heated cells because 41° C. can be delivered homogeneously in cells localized in heating area; 3) Improve the efficiency of delivering “magic bullet” to tumor cells, especially to hypoxic tumor cells due to the improvement of blood circulation in hypoxic areas of a solid tumor mass after heat treatment; 4) sensitize tumor cells to radiation and other anticancer agents by reducing the fraction of hypoxic cells.

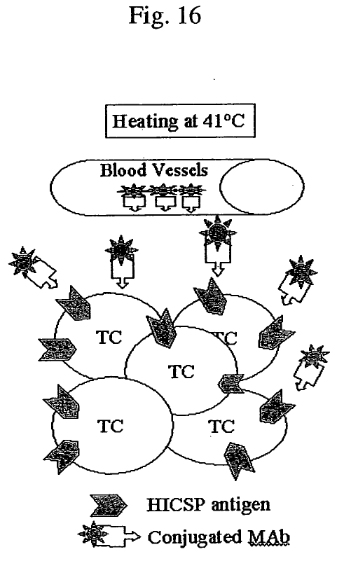

FIG. 16 illustratively depicts tumor immunotargeting therapy with heat.

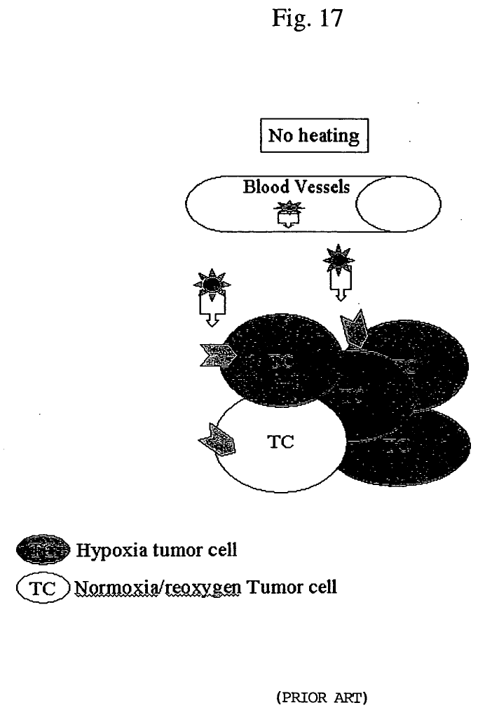

FIG. 17 is a cartoon showing prior art (tumor immunotargeting therapy without heat.

This isolated heat-inducible cell surface protein (HICSP) is believed to be important in the study of stress biology.

### SEQUENCE LIST AND IDENTIFICATION

Seq. Id. No. 1

| | |
|---|---|
| Part of novel gene sequence: | |
| TCTCCGCCTCCTTCTTCATCATGGTCATGATCCCCGGGGTGGGCTA | 408 |
| CAGC50CTTCAGGCCCACCCCAGTTCCCTCCCTACCGCGGAATGAT | |
| GCCGCCTTTC100ATGTATCCCCCATATCTCCCGTTCCCTCCGCCC | |
| TATGGACCCCAGGGGCC150TTACCGATACCCCACTCCTGATGGGC | |
| CCAGCCGTTTTCCCCGTGTGGCGG200GCCCCCGAGGCTCAGGGCC | |
| ACCAATGCGCTTAGTAGAGCCTGTGGGTCAT250CCCTCTATTCTC | |
| AAAGAGGATAATCTCAAAGAGTTTGATCAGTTGGATCA300GGAGA | |
| ATGATGATGGTTGGGCAGGGGCCCATGAAGAGGTTGACTACACTG | |
| 350AAAAGCTCAAGTTCAGCGATGAAGAAGGAGGCGGAGAGGGAGA | |
| TGTATTG400GGAGAAGG | |

Seq. Id. No. 2.

| | |
|---|---|
| Peptide sequence of HICSP60 protein | |
| SPPPSSSWS*SPGWATAFRPTPVPSLPRNDAAFHVSPISPVPSALW | 50 |
| TPGA | |
| LPIPHS*WAQPFSPCGGPPRLRATNALSRACGSSLYSQRG*SQRV* | 100 |
| SVGS | |
| GE**WLGRGP*RG*LH*KAQVQR*RRRRRGRCIGRR | |
| Total length = | 136 |

Seq. Id. No. 3.

siRNA sequence is
“tgcgcttagtagagcctgt”

### Making A Functional Living Transgenic Cell Or Mouse Useful To Practice This Discovery

In an embodiment, a transgenic organism is provided. As used herein, the term “transgenic organism” includes those transgenic organisms which contain stably integrated recombinant DNA in its cells. The transgenic organism has a new piece of DNA spliced into a chromosome in each of its cells. This “new piece” of DNA typically contains a gene that was obtained from another organism, and which has been modified so that it is expressed in the new organism. The DNA is incorporated into a vector which integrates into the host genome. The transgenic construct will contain other compounds that aid expression, stability and integration of the construct into the genome. Transgenic organisms can serve as models which are utilized in the discovery of and identification of potentially useful drugs and moieties. In an aspect a transgenic cell or transgenic mouse is provided wherein a gene comprising a polynucleotide having Seq Id No. 1 is competently integrated into the genome of the cell or of the mouse.

There are several for gene delivery into living animals such as in a living mouse. The simplest method is the direct introduction of therapeutic DNA into target cells. This approach is limited in its application because it can be used only with certain tissues and requires large amounts of DNA. The direct approach may involve use of the gene gun. In an aspect the novel gene herein is competently integrated into the transgenic animals.

As used herein the term “transgenic animal” is a mouse or a living nonhuman animal that carries a foreign gene that has been deliberately inserted into its genome. The foreign gene is constructed using recombinant DNA methodology.

One can transform embryonic stem cells (ES cells) growing in tissue culture with the desired DNA and injecting the desired gene into the pronucleus of a fertilized mouse egg and use a pronucleus method.

In addition to a structural gene, DNA usually includes other sequences to enable it to be incorporated into the DNA of the host and to be capably expressed correctly by the cells of the host. Transgenes should include promoter, enhancer, gene to be expressed, splice donor and acceptor and intron sequences, and termination/polyadenylation sequences. Transgenes must be excised from the bacterial plasmid sequences in order to be expressed in mice.

Using recombinant DNA methods, the DNA containing the structural gene is vectored into the mouse genome to enable the molecules to be inserted into the host DNA molecules. One should use promoter and enhancer sequences to enable capable gene expression by host cells. Host cells are transformed in culture by exposing cultured cells to the DNA so that some cells will incorporate it. One then selects for successfully transformed cells and injects these cells into the inner cell mass of mouse blastocysts. Embryo transfer is carried out by preparing a pseudopregnant mouse (by mating a female mouse with a vasectomized male). The stimulus of mating elicits hormonal changes needed to make the uterus of the female mouse receptive. Embryos are transferred into the uterus wherein they implant successfully and develop into healthy pups. Offspring are tested by removing a small piece of tissue from the tail and examining its DNA for the desired gene by Southern blotting. Typically, 10-20% of pups will be heterozygous for the gene. Two heterozygous mice are mated. Screening of their offspring should result in 1:4 homozygous for the transgene. Mating these will find the transgenic strain.

In an aspect the DNA is prepared as outlined above but additionally one transforms fertilized eggs in that freshly fertilized eggs are harvested before the sperm head has become a pronucleus. One then injects the male pronucleus with this DNA. When the pronuclei have fused to form the diploid zygote nucleus, one allows the zygote to divide by mitosis to form a 2-cell embryo and then implants the embryos in a pseudopregnant foster mother and proceeds as recited above.

Particle Gun Technology

Particle gun technology may be employed as a delivery system and a means to introduce desired DNA materials into cells. The particle guns shoot DNA-coated materials into living cells providing direct deposit of genetic material into living cells, intact tissues, and microscopic organelles. Du Pont Co., DuPont Building, 1007 Market Street, Wilmington, Del. 19898, USA., and Agracetus Inc., 8520 University Green, Middleton Wis. 53562, USA, have United States patent applications pending. Useful nonlimiting examples of particle guns includes the Biolistic gene gun and the Particle Gun.

Those of skill in the art after reading this specification and claims will be able to prepare a living transgenic mouse including the genomic features of this discovery. A transgenic company is genoway, Immeuble Chateaubriand, 181 avenue Jean-Jaures, 69007 LYON, France is reported to make transgenic mice.

This invention provides a novel approach for treatment of cancer involving hyperthermia and immunotargeted therapy including cytotoxic directed tumor therapy.

The invention means the HICSP target protein can be manipulated to be expressed and/or over expressed on the surface of heated cells by heating. For example purposely heat the tumor area at 41° C. for 1 hour then the HICSP expressed and/or over expressed only limited to the heated area, but not unheated area. Thus the specificity to the target (tumor cells) is much more improved over the presently available target molecules used in present tumor immunotargeting therapy.

In summary, this discovery is a novel approach for hyperthermia-based immunotargeting therapy and for the treatment of cancer and precancerous lesions.

This discovery has the following advantages as it: 1) increases specificity of biological marker to tumor cells; 2) enhances antigen expression homogeneously on heated tumor cells; 3) sensitizes tumor cells to radiation by reducing hypoxic fraction of tumor cells; and 4) improves the efficiency of delivering “magic bullet” (antibody conjugated with radio isotopes ) to tumor cells, especially hypoxic tumor cells due to the improvement of blood circulation and increase permeability of vascular in hypoxic areas of a solid tumor mass after heat treatment.

While the invention has been described in terms of various specific embodiments, those skilled in the art will recognize that the invention can be practiced with modification within the spirit and scope of this discovery.

1. An isolated, purified and characterized gene comprising a polynucleotide having a sequence depicted in Seq. Id. No. 1.

2. An isolated, purified and characterized gene having a shared homology of about at least 75% with that of the gene of claim 1.

3. An isolated, purified and characterized gene of claim 1 which is expressed by the application of heat to a living organism having the gene competently integrated in its genome.

4. A gene in accordance with claim 3 wherein a living organism is a living human cell.

5. An isolated, purified (peptide) and characterized protein comprising a polypeptide having a sequence depicted in Seq. Id. Nos. 2 & 4-12, respectively.

6. An isolated, purified and characterized protein in accordance with claim 5 having a shared homology of at least about 75% with that of said protein of claim 5.

7. An isolated, purified and characterized protein in accordance with claim 6 wherein the polypeptide is a biomarker for the presence of tumor cells in a living organism.

8. An isolated, purified and characterized protein in accordance with claim 7 wherein the protein is expressed on a cell surface or is translocated to a cell surface.

9. A protein in accordance with claim 8 wherein the protein is human protein.

10. A method of activating a gene having a Seq Id No. 1 or inducing production of a gene encoded protein therefrom in a living mammal, which comprises intentionally applying finite heat to living cells, tissues, organs or a whole body for a time and in an amount effective to cause activation of a gene comprising a polynucleotide having a sequence shown in Seq. Id. No. 1 and production of an encoded protein comprising a polypeptide having a sequence shown in Seq. Id. Nos. 2 & 4-12, respectively.

11. A method in accordance with claim 10 wherein a living mammal is a living human.

12. A method in accordance with claim 11 wherein the protein is expressed on or translocated to a cell surface.

13. A method of noninvasively activating a gene or inducing production of a gene encoded protein comprises applying an effective stressing inducing amount of an agent selected from the group consisting of mammalian stress causing chemicals and biological substance to at least one of living cells, tissues, organs or a whole living mammalian body to cause activation of a gene comprising a polynucleotide having a sequence shown in Seq. Id. No. 1 and production of an encoded protein comprising a polypeptide having a sequence shown in Seq. Id. Nos. 2 & 4-12, respectively.

14. A method in accordance with claim 13 wherein living cells comprise living human cells.

15. A method in accordance with claim 14 wherein the application of an effective stress inducing amount of an agent is intentional.

16. A method in accordance with claim 15 wherein the agent is heat energy.

17. A method in accordance with claim 16 wherein the protein is expressed on a cell surface.

18. A recombinant or transfected cell having a gene competently integrated in its genome comprising a polynucleotide having a sequence shown in Seq. Id. No.1

19. A cell in accordance with claim 18 wherein the gene encodes a protein comprising a polypeptide having a sequence shown in Seq. Id. Nos. 2 & 4-12, respectively.

20. A cell in accordance with claim 19 wherein the protein is expressed on a cell surface.

21. A biological marker useful for specifying the location of a tumor locus in tissue comprises a gene comprising a polynucleotide having a sequence shown in Seq. Id. No. 1.

22. A biomarker in accordance with claim 21 comprising a gene comprising a polynucleotide having a sequence shown in a sequence shown in Seq. Id. No. 1 is used as a tumor locating agent in a mammal.

23. A biomarker in accordance with claim 22 wherein the biomarker is a marker for cancer.

24. A biomarker in accordance with claim 23 wherein the biomarker is detected in a living mammal.

25. A biomarker in accordance with claim 24 wherein the biomarker is selectively expressed on a cell surface

26. A biological marker useful for specifying the location of a tumor locus in tissue comprises a protein comprising a polypeptide having a sequence shown in Seq. Id. Nos. 2 & 4-12, respectively.

27. A biomarker in accordance with claim 26 wherein the marker is expressed as a protein.

28. A biomarker in accordance with claim 27 wherein the protein is expressed on a cell surface.

29. A biomarker in accordance with claim 28 wherein the protein comprising a polypeptide having Seq. ID Nos. 2 & 4-12.

30. An isolated, purified and characterized antibody binding to a protein comprising a polypeptide having a sequence shown in Seq. Id. Nos. 2 & 4-12, respectively as a tumor locating agent in a living mammal.

31. An antibody in accordance with claim 30 wherein the antibody binds to a protein expressed on a cell surface.

32. A non-invasive method of using products of a gene comprising a polynucleotide having a sequence shown in Seq. Id. No. 1 as an indicator of the effectiveness of administered therapy to a mammal in the treatment of benign and malignant tumors and precancerous lesions comprises applying heat to a tissue locus containing a suspected tumor in an amount and for amount of time effective to activate the gene and forming a treated tissue locus, administering a therapy to the mammal, obtaining a sample of the locus and analyzing the sample for the extent of presence of a protein comprising a polypeptide having a sequence shown in Seq. Id. Nos. 2 & 4-12, respectively and determining that the extent provides an indication of the therapy effectiveness of the administered therapy.

33. A method in accordance with claim 32 wherein intentional heat energy is applied to a living mammal under conditions sufficient to induce hypothermia in the mammal and wherein the therapy comprises a candidate drug and further wherein the effectiveness of the drug is determined based on a detection of the extent of beneficial therapy.

34. A method in accordance with claim 33 wherein the core tumor or body temperature of the mammal is in the range from about 40° C. to about 41° C.

35. A method in accordance with claim 34 wherein the core tumor or body temperature is held at an elevated level for a time in the range from about 1 hr to about 4 hr.

36. An antibody comprising at least one of a monoclonal and polyclonal antibody recognizing a protein comprise a polypeptide having a sequence shown in Seq. Id. Nos. 2 & 4-12, respectively.

37. An antibody in accordance with claim 36 wherein the antibody is monoclonal.

38. An antibody in accordance with claim 37 wherein the antibody is polyclonal.

39. An antibody in accordance with claim 38 which binds to a protein having Seq. Id. Nos. 2 & 4-12, respectively.

40. A conjugated antibody recognizing a protein comprise a polypeptide having a sequence shown in Seq. Id. Nos. 2 & 4-12, respectively and further comprises an anti-cancer agent.

41. An antibody in accordance with claim 40 wherein the antibody binds to a protein expressed on a cell surface.

42. An antibody in accordance with claim 42 wherein the protein has Seq. Id. Nos. 2 & 4-12, respectively

43. A method of diagnosing cancer by detecting or intentionally inducing the expression of a protein comprising a polypeptide having a sequence shown in Seq. Id No 2 from a cell within a tissue locus which comprises treating a suspect benign and malignant tumor and precancerous lesion containing tissue locus in a hyperthermic manner sufficient to induce expression of a protein having a sequence shown in Seq. Id. Nos. 2 & 4-12, respectively determining if the protein is present in a sample from the tissue and if the protein is detectably present, determining that benign and malignant tumors and precancerous lesions are likely present in the tissue locus.

44. A method in accordance with claim 43 wherein intentional heat energy is applied to a living mammal under conditions sufficient to induce hypothermia in the mammal.

45. A method of treating cancer comprises administering a lethally effective amount of anti-cancer agent comprising a conjugated antibody binding to a tissue locus presenting as a target thereto a protein comprising a polypeptide having a sequence shown in Seq. Id. Nos. 2 & 4-12, respectively in a tissue locus.

46. A method in accordance with claim 45 wherein the protein is expressed on a cell surface.

47. A method in accordance with claim 46 wherein the protein is induced to express at an expression level significantly above its normal background state by the application of heat to a patient sufficient to induce safe hyperthermia.

48. A pharmaceutical composition comprising a vaccine containing protein comprising a polypeptide having a sequence shown in Seq. Id. Nos. 2 & 4-12, respectively optimally with a suitable pharmaceutically acceptable carrier.

49. A pharmaceutical kit comprises a container housing an antibody which recognizes a protein comprising a polypeptide having sequence shown in Seq. Id. Nos. 2 & 4-12, respectively and optionally a suitable pharmaceutical carrier.

50. A kit in accordance with claim 49 comprising a second antibody.

51. A kit in accordance with claim 50 wherein the kit further comprises a buffer washing component.

52. A method of treating a living mammal which comprises administering an anti-tumor agent or a functional derivative thereof, alone or in combination with a tumor-specific antibody or other tumor-directing agent to the mammal based upon the presence of a protein comprising a polypeptide having sequence shown in Seq. Id. Nos. 2 & 4-12, respectively.

53. A method in accordance with claim 52 wherein the protein is expressed on a cell surface.

54. A method in accordance with claim 53 wherein the protein is induced to express at an expression level above its normal background state by the application of heat to a patient sufficient to induce hyperthermia.

55. A method for expressing a protein having a sequence shown in Seq. Id. Nos. 2 & 4-12, respectively comprises a vector containing a gene comprising a polynucleotide having as a sequence a sequence shown in Seq. Id. No. 1.

56. A method in accordance with claim 55 wherein the protein is induced to express by the application of heat energy to a mammal under conditions and time sufficient to induce that protein.

57. A method in accordance with claim 56 wherein the protein is expressed on the surface of a cell comprising cancer.

58. A genetically engineered expression vector comprises a gene or part of sequence of the gene comprising a polynucleotide having a sequence shown in Seq. Id. No. 1.

59. An expression vector in accordance with claim 58 further comprising a transfection agent.

60. An isolated and characterized gene encoding a protein comprising a polypeptide having a sequence shown in Seq. Id. Nos. 2 & 4-12, respectively.

61. An isolated and characterized gene in accordance with claim 60 wherein the gene is operatively linked to a promoter element.

62. A engineered human antibody that binds to or reacts with a protein comprises a polypeptide having a sequence shown in Seq. Id. Nos. 2 & 4-12, respectively.

63. An antibody in accordance with claim 62 wherein the protein is expressed on a cell surface.

64. An oligo comprises an oligo synthesized according to the sequence of the gene comprising a polynucleotide having sequence shown in Seq. Id. No. 1.

65. An oligo in accordance with claim 64 wherein the oligo is double-stranded and is a siRNA.

66. An antisense oligo comprises an oligo based on a gene comprising a polynucleotide having a sequence shown in Seq. Id. No. 1.

67. An oligo in accordance with claim 66 wherein the oligo is single or double stranded.

68. A siRNA targeted to a gene comprises a polynucleotide having sequence shown in Seq. Id. No. 1.

69. An siRNA in accordance with claim 68 wherein the sequence is expressed on a cell surface.

70. A genetically engineered expression vector comprises an antisense oligo based on a polynucleotide having a sequence shown in Seq. Id. No. 1.

71. An expression vector in accordance with claim 70 wherein the vector is accompanied by a transfection agent.

72. A method of medically treating a mammal comprises administering a toxic tumor therapy to a tumor locus of the mammal, comprising administering a therapeutically effective amount of an oligo comprising a sequence of the gene comprising a polynucleotide having as a sequence a sequence shown in Seq. Id. No. 1 to the mammal.

73. A method in accordance with claim 72 wherein the oligo is transfected or infected to the tumor locus and attack cells with the active gene comprising polynucleotide having sequence shown in Seq. Id. No. 1.

74. A method in accordance with claim 73 wherein the gene expresses a protein on a cell surface.

75. A method of medically treating a mammal comprises administering a toxic tumor therapy to a tumor locus of the mammal, comprising administering a therapeutically effective amount of an antisense oligo based a gene comprising a polynucleotide having as a sequence a sequence shown in Seq. Id. No. 1 to the mammal.

76. A method in accordance with claim 75 wherein the oligo is transfected or infected to the tumor locus and attacks the gene comprising a polynucleotide having as a sequence a sequence shown in Seq. Id. No. 1.

77. A method in accordance with claim 76 wherein the tumor is cancerous.

78. A method of down and up regulating a gene comprising a polynucleotide having a sequence shown in Seq. Id. No. 1 and its encoded protein comprising a polypeptide having a sequence shown in Seq. Id. Nos. 2 & 4-12, respectively in control of tumor cell grow, invasion and metastasis.

79. A method in accordance with claim 78 wherein the sequence is expressed on the surface of a cell.

80. A pharmaceutical composition useful for treating cancer is provided comprising an oligo comprising a part of sequence of the gene comprising a polynucleotide having sequence shown in Seq. Id. No. 1 with a suitable pharmaceutically acceptable carrier.

81. A pharmaceutical composition in accordance with claim 80 which further comprises an effective amount of an anti cancer agent.

82. A pharmaceutical kit useful for treating cancer comprises a container housing an oligo having sequence of the gene comprising polynucleotide having sequence shown in Seq. Id. No. 1.

83. A kit in accordance with claim 82 which further comprises a suitable pharmaceutical carrier nontoxic to the patient.

84. A kit in accordance with claim 83 wherein the carrier comprises water or saline.

85. A method of noninvasively activating a gene or inducing production of the gene encoded protein comprises applying finite heat to living cells, tissues, organs or a whole body for a time and in an amount effective to cause activation of a gene comprising a polynucleotide having a sequence shown in Seq. Id. No. 1 and production of an encoded protein comprising a polypeptide having a sequence shown in Seq. Id. Nos. 2 & 4-12, respectively.

86. A method of noninvasively activating a gene or intentionally inducing production of the gene encoded protein comprises applying an effective stressing inducing amount of an agent selected from the group consisting of mammalian stress causing chemicals and biological substance to living cells, tissues, organs or a whole living mammalian body to cause activation of a gene comprising a polynucleotide having a sequence shown in Seq. Id. No. 1 and production of an encoded protein comprising a polypeptide having a sequence shown in Seq. Id. Nos. 2 & 4-12, respectively.

87. A cell research model comprising recombinant or transfected cells competently integrated in its genome comprising a gene further comprising a polynucleotide having a sequence shown in Seq. Id. No. 1 useful as a cell model for the function study of a gene.

88. A model in accordance with claim 87 wherein the gene encodes a protein comprising a polypeptide having a sequence shown in Seq. Id. Nos. 2 & 4-12, respectively.

89. A living biological marker useful for specifying the location of a tumor locus in tissue comprises a gene comprising a polynucleotide having a sequence shown in Seq. Id. No. 1.

90. A marker in accordance with claim 89 wherein the gene comprises a polynucleotide having a sequence shown in a sequence shown in Seq. Id. No. 1 is used as a tumor-locating agent in a mammal.

91. A marker in accordance wit claim 90 wherein the sequence is expressed on the surface of a cell.

92. A non-invasive method of using products of a gene comprising a polynucleotide having a sequence shown in Seq. Id. No. 1 as an indicator of the effectiveness of administered therapy to a mammal in the treatment of cancer comprises applying heat to a tissue locus containing a suspected tumor in an amount and for amount of time effective to activate the gene and forming a treated tissue locus, administering a therapy to the mammal, obtaining a sample of the locus and analyzing the sample for the extent of presence of a protein comprising a polypeptide having a sequence shown in Seq. Id. Nos. 2 & 4-12, respectively and determining that the extent provides an indication of the therapy effectiveness of the administered therapy.

93. Antibodies comprising monoclonal and polyclonal antibodies recognizing a protein comprise a polypeptide having a sequence shown in Seq. Id. Nos. 2 & 4-12, respectively.

94. A conjugated antibody recognizing a protein comprise a polypeptide having a sequence shown in Seq. Id. Nos. 2 & 4-12, respectively and further comprises an anti-cancer agent.

95. A method of diagnosing cancer by detecting or inducing the expression of a protein comprising a polypeptide having a sequence shown in Seq. Id No 2 from a cell within a tissue locus which comprises treating a suspect tumor containing tissue locus in a hyperthermic manner sufficient to induce expression of a protein having a sequence shown in Seq. Id. Nos. 2 & 4-12, respectively, determining if the protein is present in a sample from the tissue and if the protein is detectably present, determining that cancer is likely present in the tissue locus.

96. A method of treating cancer comprises administering a lethally effective amount of anti-cancer agent comprising a conjugated antibody binding to a tissue locus presenting as a target thereto a protein comprising a polypeptide having a sequence shown in Seq. Id. Nos. 2 & 4-12, respectively in a tissue locus.

97. A method in accordance with claim 98 wherein the locus of the target is determined by the method of claim 1.

98. A method of treating a living mammal which comprises administering an anti-tumor agent or a functional derivative thereof, alone or in combination with a tumor-specific antibody or other tumor-directing agent to the mammal based upon the presence of a protein comprising a polypeptide having sequence shown in Seq. Id. Nos. 2 & 4-12, respectively.

99. A method in accordance with claim 98 wherein the protein is expressed on a cell surface and is induced by intentional hyperthermia.

100. A method for expressing a protein having a sequence shown in Seq. Id. Nos. 2 & 4-12, respectively comprises a vector containing a gene comprising a polynucleotide having as a sequence a sequence shown in Seq. Id. No. 1.

101. A method in accordance with claim 100 wherein the protein is expressed on a cell surface and is induced by intentional hyperthermia.

102. A genetically engineered expression vector comprises a gene or part of sequence of the gene comprising a polynucleotide having a sequence shown in Seq. Id. No. 1.

103. A method in accordance with claim 102 wherein the gene encodes a protein comprising a polypeptide having a sequence shown in Seq. Id. Nos. 2 & 4-12, respectively. In an aspect, the gene is operatively linked to a promoter element.

104. An engineered human antibody that binds to or reacts with a protein comprises a polypeptide having a sequence shown in Seq. Id. Nos. 2 & 4-12, respectively.

105. An antibody in accordance with claim 104 wherein the protein is expressed on the cell surface.

106. An oligo comprises an oligo synthesized according to the sequence of the gene comprising a polynucleotide having sequence shown in Seq. Id. No. 1.

107. An antisense oligo comprises an oligo based on a gene comprising a polynucleotide having a sequence shown in Seq. Id. No. 1.

108. An oligo in accordance with claim 107 wherein the oligo is single stranded.

109. An oligo in accordance with claim 107 wherein the oligo is double stranded.

110. A siRNA targeted to a gene comprises a polynucleotide having sequence shown in Seq. Id. No. 1.

111. A genetically engineered expression vector comprising an antisense oligo based on a polynucleotide having a sequence shown in Seq. Id. No. 1.

112. A method of medically treating a mammal comprises administering a toxic tumor therapy to a tumor locus of the mammal, comprising administering a therapeutically effective amount of an oligo comprising a sequence of the gene comprising a polynucleotide having as a sequence a sequence shown in Seq. Id. No. 1 to the mammal.

113. A method in accordance with claim 112 wherein the oligo is double-stranded and is an siRNA oligo.

114. A method in accordance with claim 112 wherein the oligo is transfected or infected to the tumor locus and attack cells with the active gene comprising polynucleotide having sequence shown in Seq. Id. No. 1.

115. A method of medically treating a mammal comprises administering a toxic tumor therapy to a tumor locus of the mammal, comprising administering a therapeutically effective amount of an antisense oligo based a gene comprising a polynucleotide having as a sequence a sequence shown in Seq. Id. No. 1 to the mammal.

116. A method in accordance with claim 115 wherein the oligo is transfected or infected to the tumor locus and attacks the gene comprising a polynucleotide having as a sequence a sequence shown in Seq. Id. No. 1.

117. A method of down and up regulating a gene comprising a polynucleotide having a sequence shown in Seq. Id. No. 1 and its encoded protein comprising a polypeptide having a sequence shown in Seq. Id. Nos. 2 & 4-12, respectively in control of tumor cell grow, invasion and metastasis.

118. A method in accordance with claim 117 wherein the protein is expressed on a cell surface.

119. A pharmaceutical composition comprising an oligo further comprising a part of sequence of the gene comprising a polynucleotide having sequence shown in Seq. Id. No. 1 with a suitable pharmaceutically acceptable carrier.

120. A composition in accordance with claim 119 wherein the composition further comprises a pharmaceutically acceptable carrier.

121. A pharmaceutical kit comprises a container housing an oligo having sequence of the gene comprising polynucleotide having sequence shown in Seq. Id. No. 1.

122. A kit in accordance with claim 121 further comprising a suitable pharmaceutical carrier, such as water or saline.

123. A method of detecting cancerous tissue in vivo subject, comprising providing an antibody or antigen binding portion thereof which binds to the extracellular domain of a protein having SEQ. ID. Nos. 2 & 4-12, respectively wherein the antibody or antigen binding portion thereof is bound to a label effect to permit detection of cancerous tissue, contacting the tissue with the antibody or antigen binding portion thereof under conditions effective to permit binding; and detecting the presence of cancerous tissue by detecting the label.

124. A method in accordance with claim 123 wherein detecting the label provides an indication of where cancerous tissue is localized within the body of the patient.

125. A method according to claim 124 wherein the label is detected using an imaging device.

126. A method according to claim 124, wherein the subject is a living human.

127. A method according to claim 124 wherein the administering is carried out parenterially.

128. A method according to claim 127 wherein the administering is carried out intravenously.

129. A method according to claim 127, wherein the administering is carried out by intracavitary instillation.

130. A method according to claim 127 wherein the label is a radiolabel and is an alpha emitter.

131. A method according to claim 127 wherein the radiolabel is a beta emitter.

132. A method according to claim 127 wherein the radiolabel is a gamma emitter.

133. A transgenic living mouse having competently integrated in its genome a functional genomic expression comprising the gene of claim 1.

134. A mouse in accordance with claim 133 wherein the gene expresses a protein genoming polypeptide having Seq. Id. Nos. 2 & 4-12, respectively.

135. A method in accordance with claim 134 wherein the drug is scored as to the cytoxic effectiveness toward benign and malignant tumors and precancerous lesions.

136. A method in accordance with claim 135 wherein the prioritization includes ranking the drug as to its potential cytoxicity.

137. A method in accordance with claim 136 wherein a transgenic nonhuman living mammal is prepared to carry benign and malignant tumors and precancerous lesions and carries benign and malignant tumors and precancerous lesions.

138. In an aspect, a method of reducing at least one of the size and number of tumor cells in a living mammal comprises administering to the mammal an effective amount of an antibody which binds to a protein comprising a polypeptide having sequence shown in Seq. Id. Nos. 2 & 4-12, respectively. In an aspect, the antibody is coupled to a tumor cytotoxic agent.

139. In an aspect, a method to treat patients with benign and/or malignant tumors based on stress, including hyperthermia, inducible cell surface proteins.

140. In an aspect, a method to treat patients with benign and/or malignant tumors with stress, including hyperthermia, based tumor immunotargeting therapy.

141. In an aspect, antibody against HICSP conjugated with nanoparticles that contain therapeutic reagents.

142. In an aspect, antibody against HICSP conjugated with temperature sensitive liposome encapsulated therapeutic reagents. For example the antibody against HICSP guide the liposome containing therapeutic reagents to HICSP expressing cells and release the therapeutic reagents in temperature elevated condition due to the liposome are sensitive to moderate hyperthermia ( 39° C. to 42.5° C.).

143. In an aspect, antibody against HICSP conjugated with anticancer pre-drugs that activated by hyperthermia. For example the antibody against HICSP guide the anticancer pre-drugs to HICSP expressing cells and activated by moderate hyperthermia (39° C. to 42.5° C.).

144. In an aspect, repeated heating strategy may more effective in the inducing and/or accumulating HICSP protein on the cell surface. For example heating at 41° C. for 1 hr and with 24 hr interval for multiple times.

145. In an aspect, repeated heating strategy may be used in heat activated anticancer pre-drugs and temperature sensitive liposome particles encapsulated anticancer reagents. For example heating at 41° C. for 1 hr, then administering conjugated antibody with heat activated anticancer pre-drugs or heat sensitive liposome encapsulated with anticancer reagents, and then heat again for practically suitable time.
